(19) United States
(12) Patent Application Publication
(10) Pub. No.: US 2024/0254155 A1
(43) Pub. Date: Aug. 1, 2024

(54) NOVEL COMPOUND ISOLATED FROM SPINACH, AND COMPOSITION COMPRISING SAME FOR PREVENTING OR TREATING INFLAMMATORY DISEASES

(71) Applicant: KOREA RESEARCH INSTITUTE OF BIOSCIENCE AND BIOTECHNOLOGY, Daejeon (KR)

(72) Inventors: Hyung Won RYU, Daejeon (KR); Su Ui LEE, Daejeon (KR); Sei-Ryang OH, Daejeon (KR); Jae-Won LEE, Daejeon (KR); Doo-Young KIM, Daejeon (KR); Hyun-Jae JANG, Daejeon (KR); Mun-Ock KIM, Daejeon (KR); Seong Hun JEONG, Gyeongsangnam-do (KR)

(73) Assignee: KOREA RESEARCH INSTITUTE OF BIOSCIENCE AND BIOTECHNOLOGY, Daejeon (KR)

(21) Appl. No.: 18/564,361

(22) PCT Filed: May 4, 2022
(86) PCT No.: PCT/KR2022/006445

§ 371 Date: Nov. 27, 2023

(30) Foreign Application Priority Data
May 27, 2021 (KR) 10-2021-0068641

(51) Int. Cl. C07H 15/256; A61K 31/704; A61K 36/21; A61P 11/06; A61P 29/00; C07H 1/08

(52) U.S. Cl. CPC C07H 15/256; A61K 31/704; A61K 36/21; A61P 11/06; A61P 29/00; C07H 1/08; A61K 2236/33; A61K 2236/55

(57) ABSTRACT

The present invention relates to a novel compound isolated from spinach, and a composition comprising same for preventing or treating inflammatory diseases, and, more particularly, to a novel medicagenic acid glycoside compound isolated from spinach, a preparation method therefor, and a composition comprising same for preventing or treating inflammatory diseases.

### TECHNICAL FIELD

The present application claims priority to Korean Patent Application No. 2021-0068641 filed on May 27, 2021, and the entire specification thereof is incorporated herein by reference.

The present invention relates to a novel compound isolated from spinach, and a composition comprising same for preventing or treating inflammatory diseases, and, more particularly, to a novel medicagenic acid glycoside compound isolated from spinach, a preparation method therefor, and a composition comprising same for preventing or treating inflammatory diseases.

### BACKGROUND ART

In normal cases, inflammation neutralizes or eliminates causative factors through an inflammatory response in the body and regenerates damaged tissues to restore normal structure and function. However, it becomes a problem when the degree of inflammation exceeds a certain level or becomes chronic and progresses to a disease state such as chronic inflammation. Not only can inflammatory reactions be observed in almost all clinical diseases, but enzymes related to inflammatory reactions are known to play an important role in carcinogenesis.

The progression of inflammatory reactions in the body is known to be related to COX enzyme activity, and the COX enzyme is known to comprise COX-1 and COX-2. Among these, COX-2 is an enzyme that is expressed temporarily and rapidly within cells by cell division factors (mitogens) or cytokines in the occurrence of inflammation or other immune reactions. In addition, nitric oxide (NO), a powerful inflammatory mediator, is produced from L-arginine by NO synthase (NOS). Inflammatory stimulation caused by external stress such as UV or substances such as endotoxin or cytokines increases the expression of inducible NOS (iNOS) in cells. This may induce NO production within cells to activate macrophages and cause an inflammatory response.

Meanwhile, respiratory diseases generally involve inflammation, which worsens the condition of the bronchi, lungs, etc. The physiological function of mucus present in the respiratory tract is mainly due to the physicochemical properties of mucin, a mucous glycoprotein. Mucin is a glycoprotein with a molecular weight of several million daltons, and its carbohydrate structure shows considerable diversity. When a mild disease occurs in normal physiological conditions or in respiratory clinical settings, the bio-defensive role of airway mucus is essential. However, the formation of a mucus plug due to an extreme increase in the viscosity of mucus and changes in the physicochemical properties of mucus actually hinders the discharge of airway secretions and causes bronchial obstruction due to deposited secretions and drainage difficulties in the event of infection.

Therefore, for patients with respiratory diseases who suffer great pain due to excessive secretion of airway mucus or changes in viscosity, control of airway mucus secretion is very important in alleviating pain caused by the disease and treating the disease.

### SUMMARY OF INVENTION

### Technical Problem

Therefore, the present inventor conducted extensive research to develop new therapeutic substances derived from natural products that exhibit anti-inflammatory effects and can effectively inhibit secretion of airway mucus in inflammatory respiratory diseases and, as a result, discovered that four (4) novel glycoside compounds isolated from a spinach extract and fractions comprising same showed such an effect, and completed the present invention.

Accordingly, the object of the present invention is to provide compounds represented by the following formulae 1 to 6:

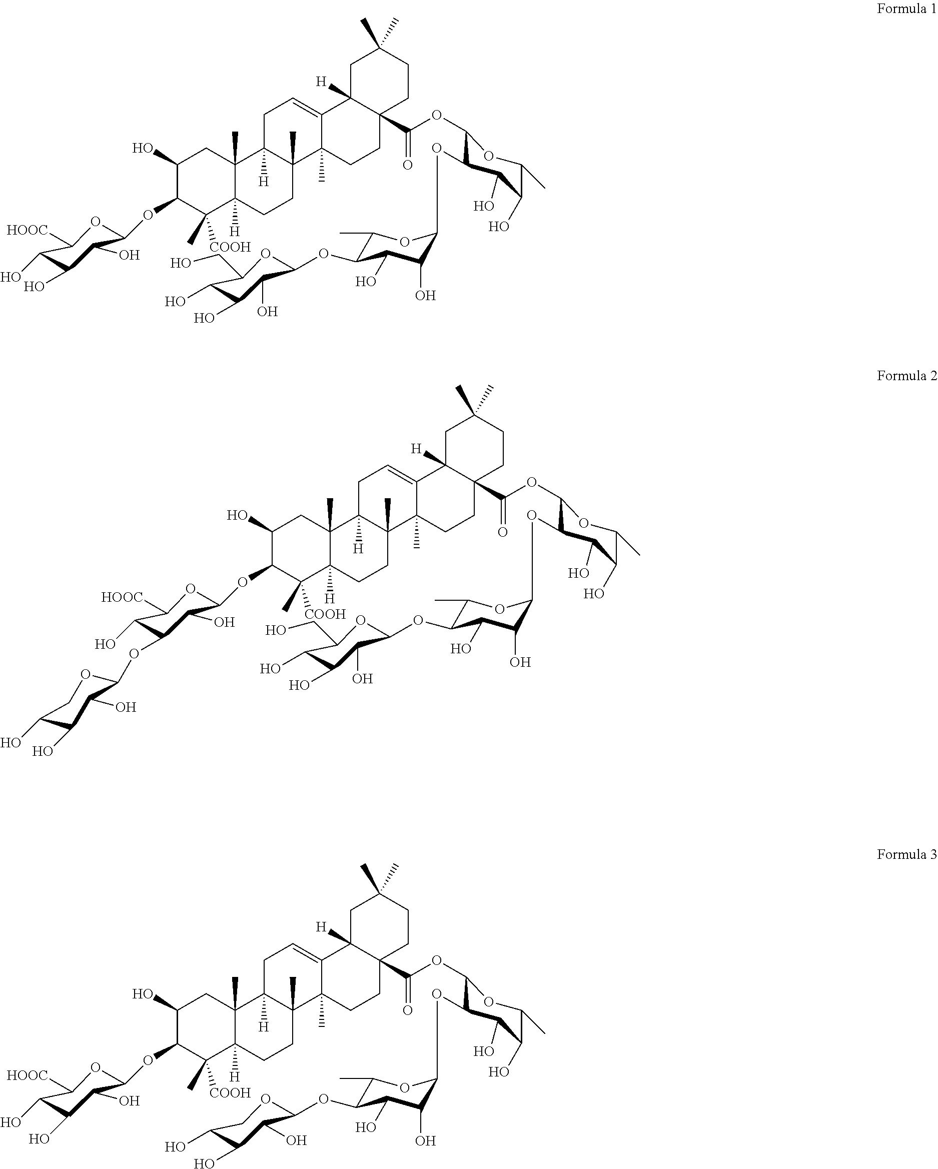

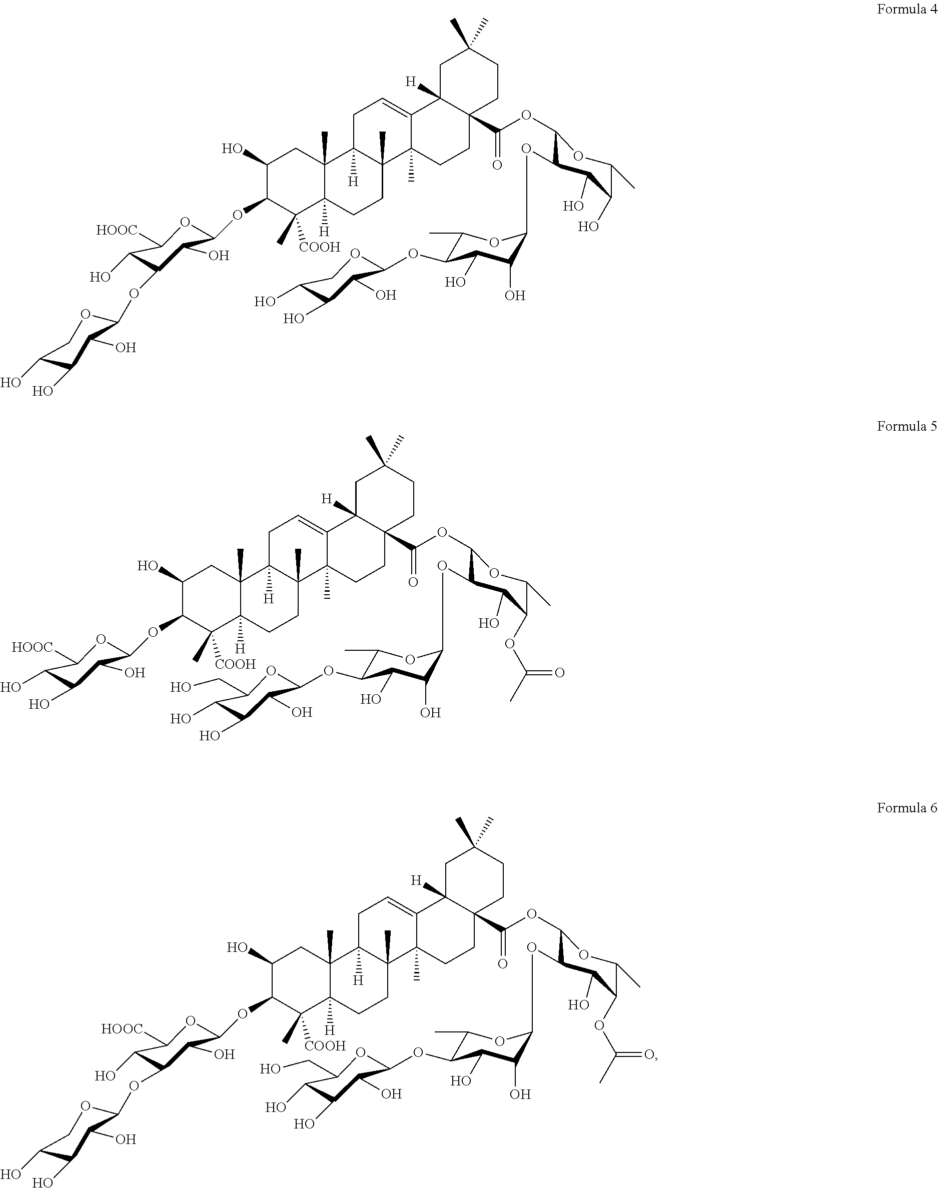

pharmaceutically acceptable salts thereof, isomers thereof, hydrates thereof, or solvates thereof.

Another object of the present invention is to provide a method for preparing the compounds of formulae 1 to 6, comprising: (a) extracting spinach with a solvent selected from the group consisting of water, organic solvents, subcritical fluids, supercritical fluids and mixtures thereof; (b) fractionating the spinach extract by chromatography to obtain fractions; and (c) isolating the compounds of the above-described formulae 1 to 6 from the fractions.

Another object of the present invention is to provide a pharmaceutical composition for preventing or treating inflammatory diseases, comprising as an active ingredient one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a pharmaceutical composition for preventing or treating inflammatory diseases, consisting of one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a pharmaceutical composition for preventing or treating inflammatory diseases, consisting essentially of one or more selected from the group consisting of the compounds represented by the above-described formulae 1 to 6, pharmaceutically acceptable salts thereof, isomers thereof, hydrates thereof, and solvates thereof.

Another object of the present invention is to provide a pharmaceutical composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

Another object of the present invention is to provide a food composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a food composition for preventing or improving inflammatory diseases consisting of one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a food composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

Another object of the present invention is to provide a food composition for preventing or improving inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

Another object of the present invention is to provide a veterinary composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a veterinary composition for preventing or improving inflammatory diseases consisting of one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a veterinary composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

Another object of the present invention is to provide a feed composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a feed composition for preventing or improving inflammatory diseases consisting of one or more selected from the group consisting of the compounds represented by the above-described formulae 1 to 6, pharmaceutically acceptable salts thereof, isomers thereof, hydrates thereof, and solvates thereof.

In addition, another object of the present invention is to provide a feed composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

Another object of the present invention is to provide use of one or more selected from the group consisting of the compounds represented by the above-described formulae 1 to 6, pharmaceutically acceptable salts thereof, isomers thereof, hydrates thereof, and solvates thereof, for preparing a composition for treating inflammatory diseases.

Another object of the present invention is to provide a method of treating inflammatory diseases comprising administering to a subject in need thereof an effective amount of a composition comprising as an active ingredient one or more selected from the group consisting of the compounds represented by the above-described formulae 1 to 6, pharmaceutically acceptable salts thereof, isomers thereof, hydrates thereof, and solvates thereof.

### Solution to Problem

In order to achieve the above-described objects of the present invention, the present invention provides compounds represented by the following formulae 1 to 6:

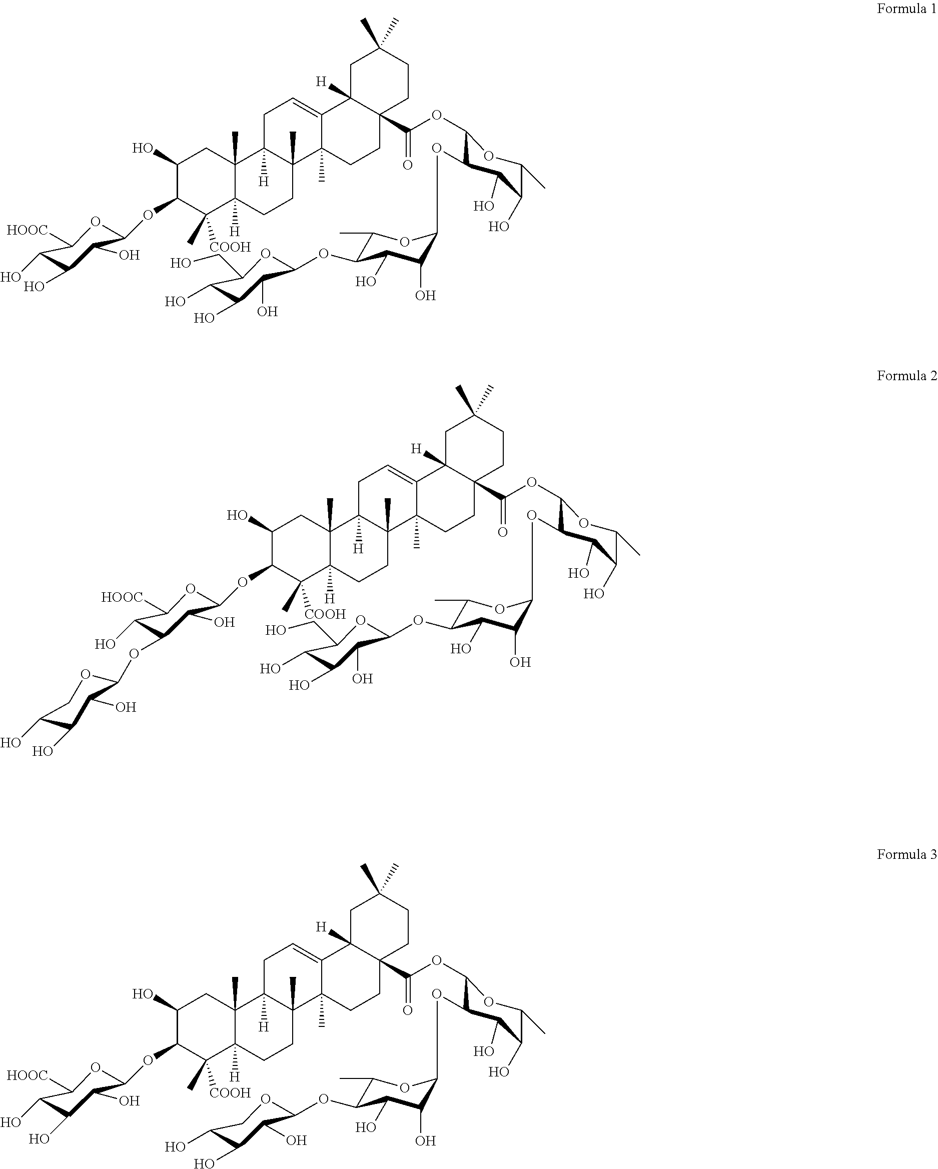

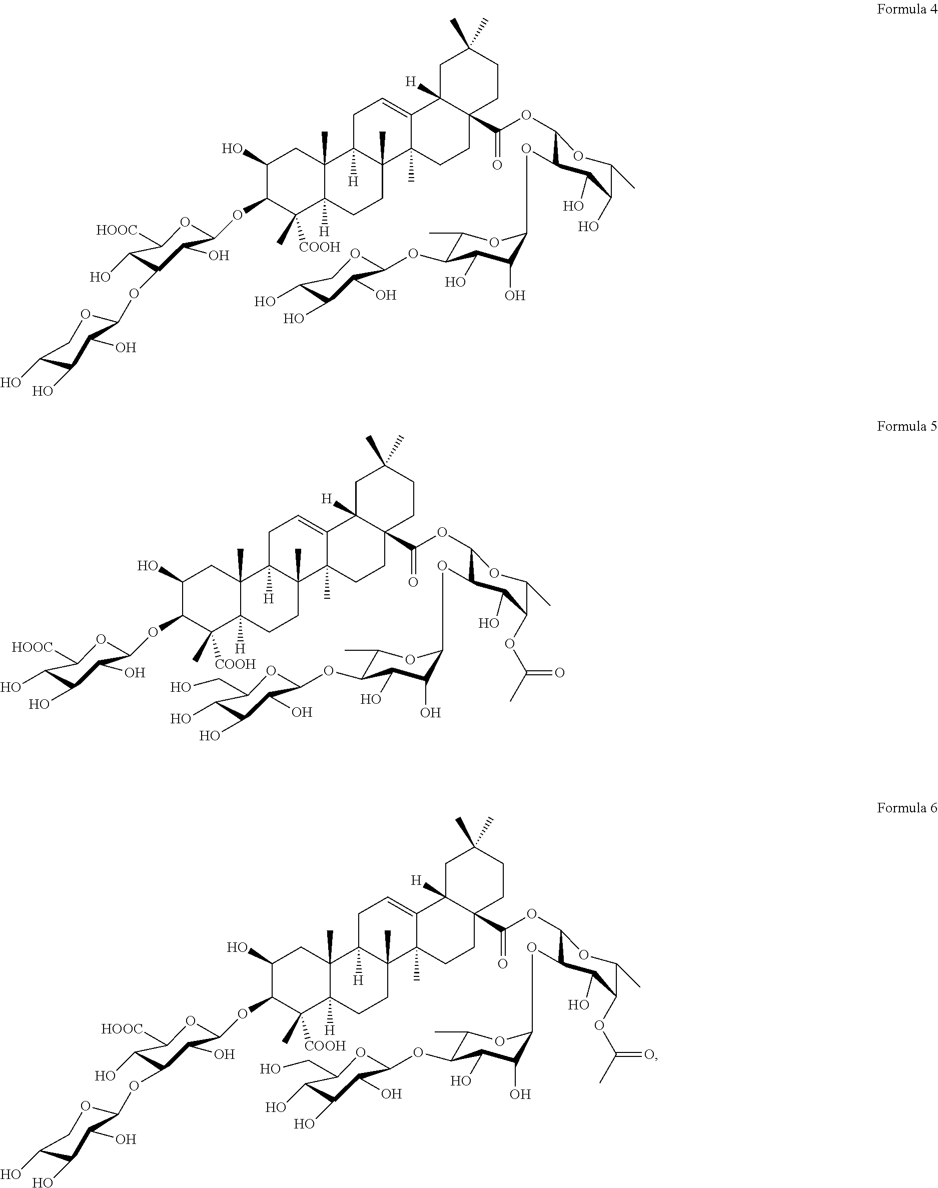

pharmaceutically acceptable salts thereof, isomers thereof, hydrates thereof, or solvates thereof.

In order to achieve another object of the present invention, the present invention provides a method for preparing the compounds of the above-described formulae 1 to 6, comprising: (a) extracting spinach with a solvent selected from the group consisting of water, organic solvents, subcritical fluids, supercritical fluids and mixtures thereof; (b) fractionating the spinach extract by chromatography to obtain fractions; and (c) isolating the compounds of formulae 1 to 6 from the fractions.

In order to achieve another object of the present invention, the present invention provides a pharmaceutical composition for preventing or treating inflammatory diseases, comprising as an active ingredient one or more selected from the group consisting of the compounds represented by the above-described formulae 1 to 6, pharmaceutically acceptable salts thereof, isomers thereof, hydrates thereof, and solvates thereof.

In addition, the present invention provides a pharmaceutical composition for preventing or treating inflammatory diseases, consisting of one or more selected from the group consisting of the compounds represented by the above-described formulae 1 to 6, pharmaceutically acceptable salts thereof, isomers thereof, hydrates thereof, and solvates thereof.

In addition, the present invention provides pharmaceutical composition for preventing or treating inflammatory diseases, consisting essentially of one or more selected from the group consisting of the compounds represented by the above-described formulae 1 to 6, pharmaceutically acceptable salts thereof, isomers thereof, hydrates thereof, and solvates thereof.

In order to achieve another object of the present invention, the present invention provides a pharmaceutical composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

In order to achieve another object of the present invention, the present invention provides a food composition for preventing or improving inflammatory diseases, comprising one or more selected from the group consisting of the compounds represented by the above-described formulae 1 to 6, pharmaceutically acceptable salts thereof, isomers thereof, hydrates thereof, and solvates thereof.

In addition, the present invention provides a food composition for preventing or improving inflammatory diseases, consisting of one or more selected from the group consisting of the compounds represented by the above-described formulae 1 to 6, pharmaceutically acceptable salts thereof, isomers thereof, hydrates thereof, and solvates thereof.

In addition, the present invention provides a food composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

In order to achieve another object of the present invention, the present invention provides a food composition for preventing or improving inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

In order to achieve another object of the present invention, the present invention provides a veterinary composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

In addition, the present invention provides a veterinary composition for preventing or improving inflammatory diseases consisting of one or more selected from the group consisting of the compounds represented by the above-described formulae 1 to 6, pharmaceutically acceptable salts thereof, isomers thereof, hydrates thereof, and solvates thereof.

In addition, the present invention provides a veterinary composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

In order to achieve another object of the present invention, the present invention provides a feed composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

In addition, the present invention provides a feed composition for preventing or improving inflammatory diseases consisting of one or more selected from the group consisting of the compounds represented by the above-described formulae 1 to 6, pharmaceutically acceptable salts thereof, isomers thereof, hydrates thereof, and solvates thereof.

In addition, the present invention provides a feed composition for preventing or improving inflammatory diseases consisting essentially of one or more selected from the group consisting of the compounds represented by the above-described formulae 1 to 6, pharmaceutically acceptable salts thereof, isomers thereof, hydrates thereof, and solvates thereof.

In order to achieve another object of the present invention, the present invention provides use of one or more selected from the group consisting of the compounds represented by the above-described formulae 1 to 6, pharmaceutically acceptable salts thereof, isomers thereof, hydrates thereof, and solvates thereof, for preparing a composition for treating inflammatory diseases.

In order to achieve another object of the present invention, the present invention provides a method of treating inflammatory diseases comprising administering to a subject in need thereof an effective amount of a composition comprising as an active ingredient one or more selected from the group consisting of the compounds represented by the above-described formulae 1 to 6, pharmaceutically acceptable salts thereof, isomers thereof, hydrates thereof, and solvates thereof.

Hereinafter, the present invention will be described in detail.

The present invention provides compounds represented by the following the above-described formulae 1 to 6:

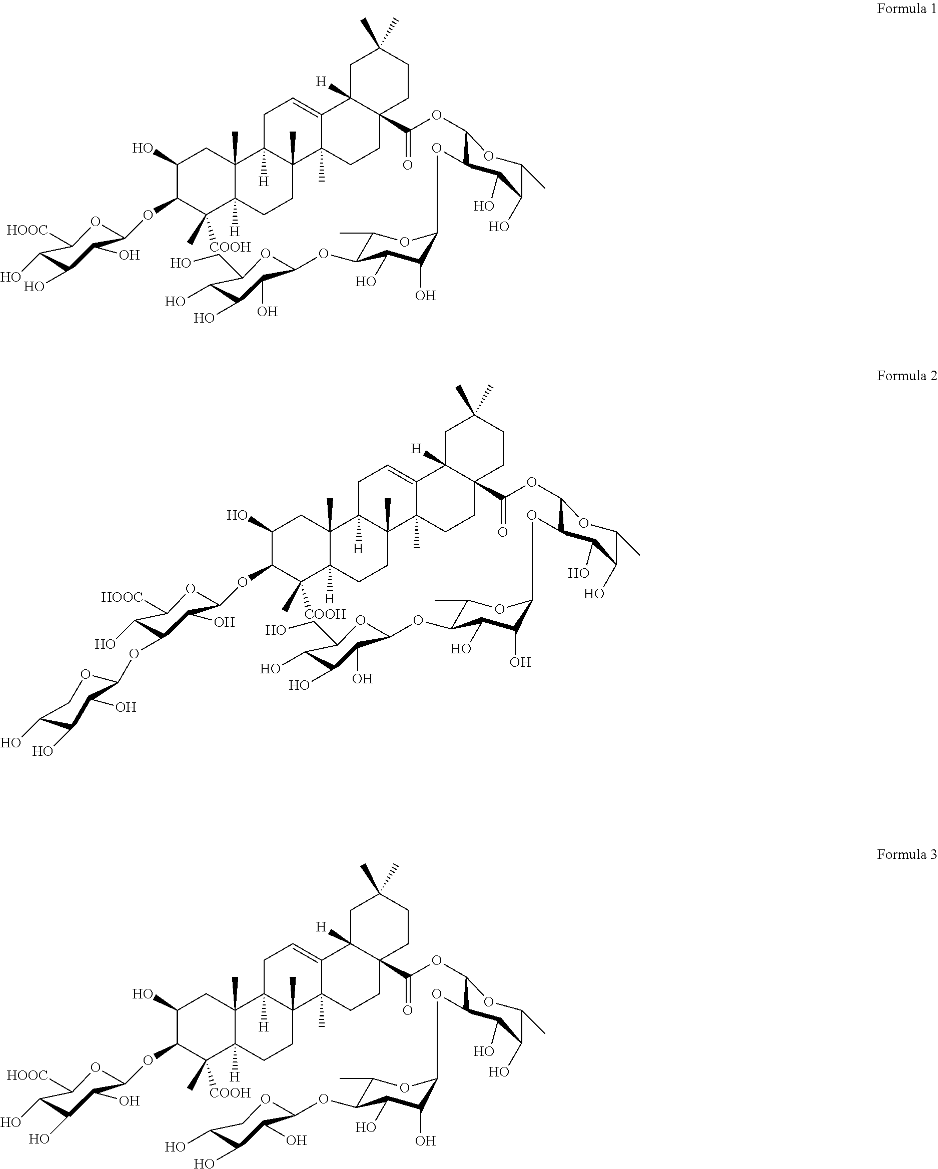

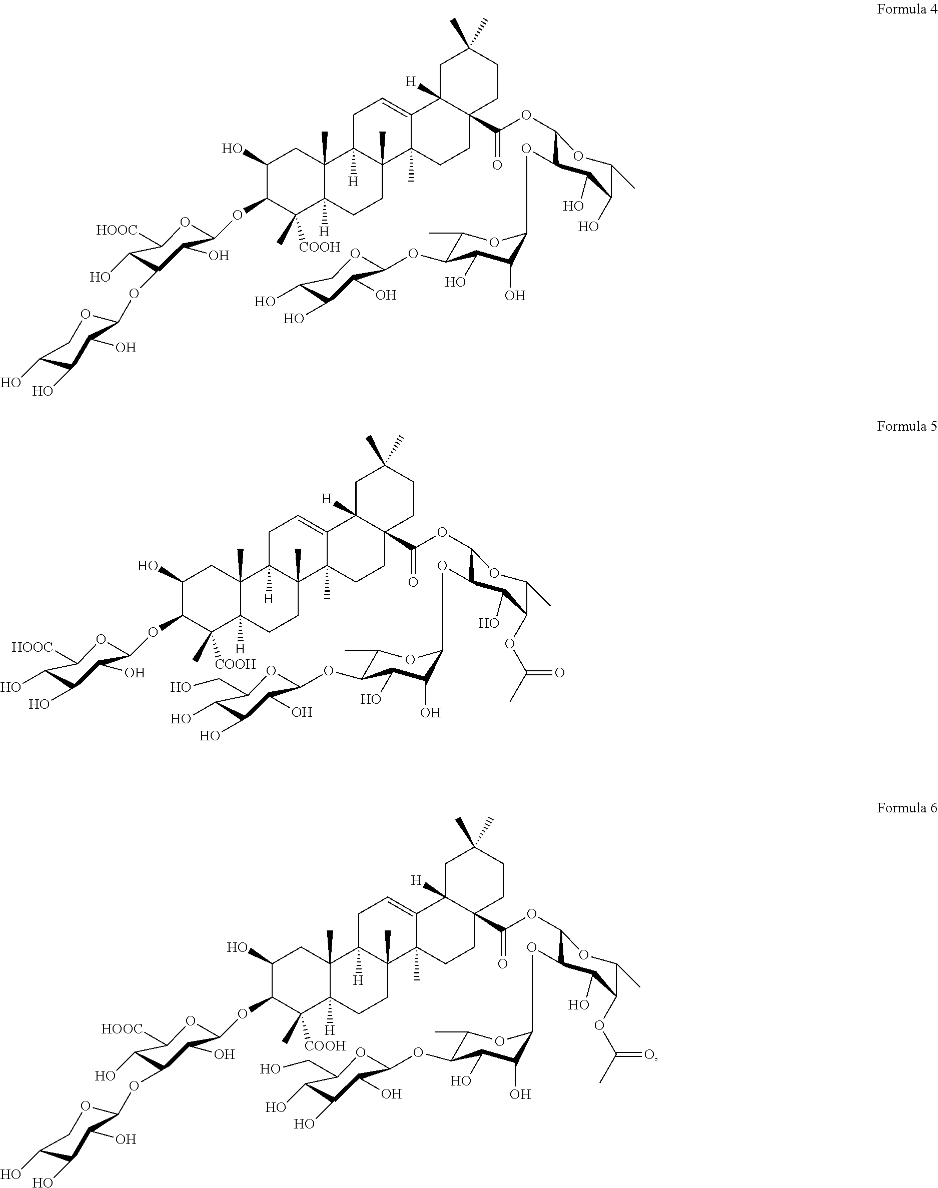

pharmaceutically acceptable salts thereof, isomers thereof, hydrates thereof, or solvates thereof.

In one embodiment of the present invention, the present inventor isolated novel glycosides of medicagenic acid represented by the above-described formulae 1 to 6 from a spinach extract. The novel glycosides represented by the above-described formulae 1 to 6 were confirmed to exhibit significantly improved anti-inflammatory efficacy, as compared to medicagenic acid, and the fractions of the spinach extract comprising same were also confirmed to exhibit significantly improved anti-inflammatory efficacy, as compared to spinach extract.

As used herein, the term “pharmaceutically acceptable” means recognized as capable of being approved by a government or equivalent regulatory agency, approved, or listed in a pharmacopoeia or other general pharmacopoeia that it can be used in animals, more specifically in human beings, by avoiding significant toxic effects when used in normal medicinal doses.

As used herein, the term “pharmaceutically acceptable salts” refer to a salt that is pharmaceutically acceptable and has the desired pharmacological activity of the parent compound. The salt may be an acid addition salt formed with a pharmaceutically acceptable free acid. Acid addition salts are obtained from inorganic acids such as hydrochloric acid, nitric acid, phosphoric acid, sulfuric acid, hydrobromic acid, hydroiodic acid, nitrous acid and phosphorous acid, and non-toxic organic acids such as aliphatic mono and dicarboxylates, phenyl-substituted alkanoates, hydroxy alkanoates and alkanedioates, aromatic acids, aliphatic and aromatic sulfonic acids. These pharmaceutically non-toxic salts comprise sulfates, pyrosulfates, bisulfates, sulfites, bisulfites, nitrates, phosphates, monohydrogen phosphates, dihydrogen phosphates, metaphosphates, pyrophosphate chlorides, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxy Benzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, hydroxybutyrate, glycolate, malate, tartrate, methanesulfonate, propanesulfonate, naphthalene-1-sulfonate, naphthalene-2-sulfonate or mandelate. However, the present invention is not limited thereto.

As used herein, the term “isomers” may comprise, as well as optical isomers (e.g., essentially pure enantiomers, essentially pure diastereomers, or mixtures thereof), conformation isomers (i.e., isomers that differ only in the angle of one or more chemical bonds), position isomers (especially, tautomers), or geometric isomers (e.g., cis-trans isomers).

As used herein, the term “essentially pure, ” when used in relation to, for example, enantiomers or partial isomers, may mean that a specific compound, exemplified by an enantiomer or a partial isomer, is present in an amount of at least about 90%, preferably at least about 95%, more preferably at least about 97% or at least about 98%, even more preferably at least about 99%, even far more preferably at least about 99.5% (w/w).

As used herein, the term “hydrate” refers to a compound to which water is bound, and may refer to a broad concept comprising embedded compounds in which there is no chemical bond between water and the compound.

As used herein, the term “solvate” may refer to a molecule of a solute, a molecule of an ion and a solvent, or a higher-order compound formed between ions.

In the present invention, the glycoside compounds represented by the above-described formulae 1 to 6 may be extracted from spinach.

The present invention provides a method for preparing the compound according to claim 1, comprising (a) extracting spinach with a solvent selected from the group consisting of water, organic solvents, subcritical fluids, supercritical fluids and mixtures thereof; (b) fractionating the spinach extract by chromatography to obtain fractions; and (c) isolating the compounds of formulae 1 to 6 from the fractions.

In the present invention, the spinach (Spinaciaoleracea L.) is an annual or biennial herb of the pigweed family of the central order of dicotyledonous plants, and the spinach extract may be a leaf, stem, flower, root, bud, whole plant, or a mixed extract thereof.

In the present invention, step (a) is a step of preparing a spinach extract by a method of extracting natural products commonly known in the art. In step (a), extraction may be performed with one or more solvents selected from the group consisting of water, organic solvents, subcritical fluids, supercritical fluids and mixtures thereof. The organic solvent having 1 to 6 carbon atoms may be selected from the group consisting of alcohol, acetone, ether, benzene, chloroform, ethyl acetate, methylene chloride, hexane, cyclohexane and petroleum ether having 1 to 6 carbon atoms. However, the present invention is not limited thereto. If the extraction solvent in step (a) is a mixed solvent of water and organic solvent, water and organic solvent may be mixed in a ratio of 90:10 to 10:90 and, specifically, in a ratio of 90:10, 80:20, 70:30, 60:40, 50:50, 40:60, 30:70, 20:80 or 10:90.

In the present invention, step (b) is a step of isolating fractions comprising the novel glycosides of the above-described formulae 1 to 6 from the spinach extract prepared in step (a).

In the present invention, the type of chromatography method is not particularly limited, and any method used in the art to isolate or purify the desired component may be used without limitation. The chromatographic method may preferably be column chromatography and, more preferably, be medium pressure liquid chromatography (MPLC), high-performance liquid chromatography (HPLC), or ultra-high-performance liquid chromatography (UHPLC).

In step (b), the chromatographic method may be performed 1 to 5 times considering yield and purity, but the present invention is not limited thereto.

The mobile phase or developing solvent in the process of fractionating the spinach extract by chromatography in step (b) may comprise water, an organic solvent, or a mixed solvent thereof, and, preferably, a mixed solvent of water and a non-polar solvent.

The type of the non-polar solvent is not particularly limited, but is preferably methanol or acetonitrile, and, most preferably, methanol.

In step (b), the chromatography may be sequentially

developed according to a concentration gradient by using water and a non-polar solvent as mobile phases, and the ratio of the water and the non-polar solvent may be sequentially adjusted from 100:0 to 0:100 to perform the chromatography. Specifically, chromatography may be performed by using water and a nonpolar solvent as mobile phases in a ratio of 100:0, 90:10, 80:20, 70:30, 60:40, 50:50, 40:60, 30:70, 20:80, 10:90 or 0:100.

Step (b) of the present invention may be characterized in that, when fractionation of the spinach extract is carried out by sequentially developing the chromatography according to a concentration gradient by using water and a non-polar solvent as mobile phases, fractions fractionated in a 20 to 100% (v/v) of an aqueous solution of a non-polar solvent, preferably in a 30 to 100% (v/v) of an aqueous solution of a non-polar solvent, are obtained.

According to one embodiment of the present invention, it was confirmed that, when the spinach extract was fractionated by chromatography by using a mixed solvent of water and methanol, the novel glycoside compounds of the above-described formulae 1 to 6 were contained in 30 to 100% (v/v) of the methanol aqueous solution fraction.

In the present invention, step (c) is a step of isolating the desired new glycosides of formulae 1 to 6 from the fractions of the spinach extract obtained in step (b).

The present invention provides a pharmaceutical composition for preventing or treating inflammatory diseases, comprising as an active ingredient one or more selected from the group consisting of the compounds represented by the following formulae 1 to 6:

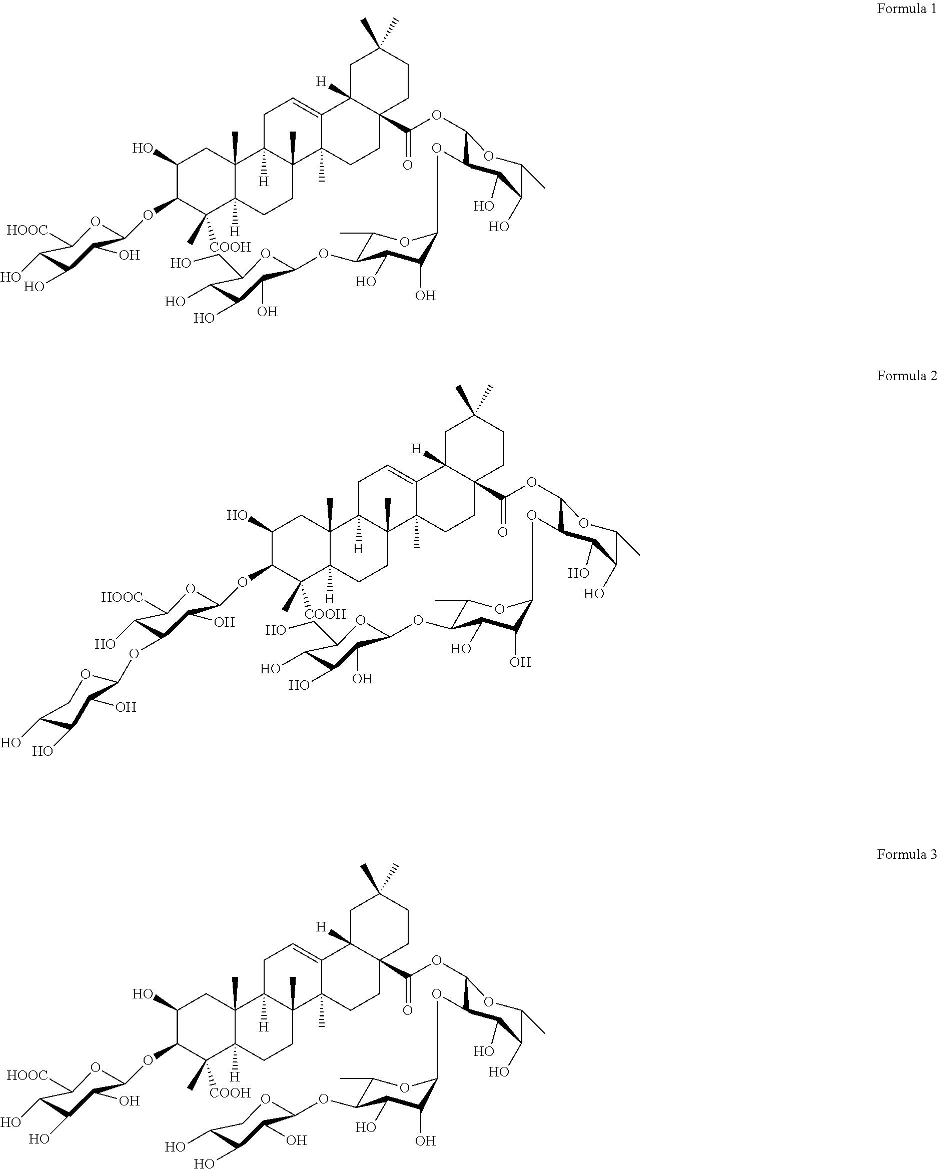

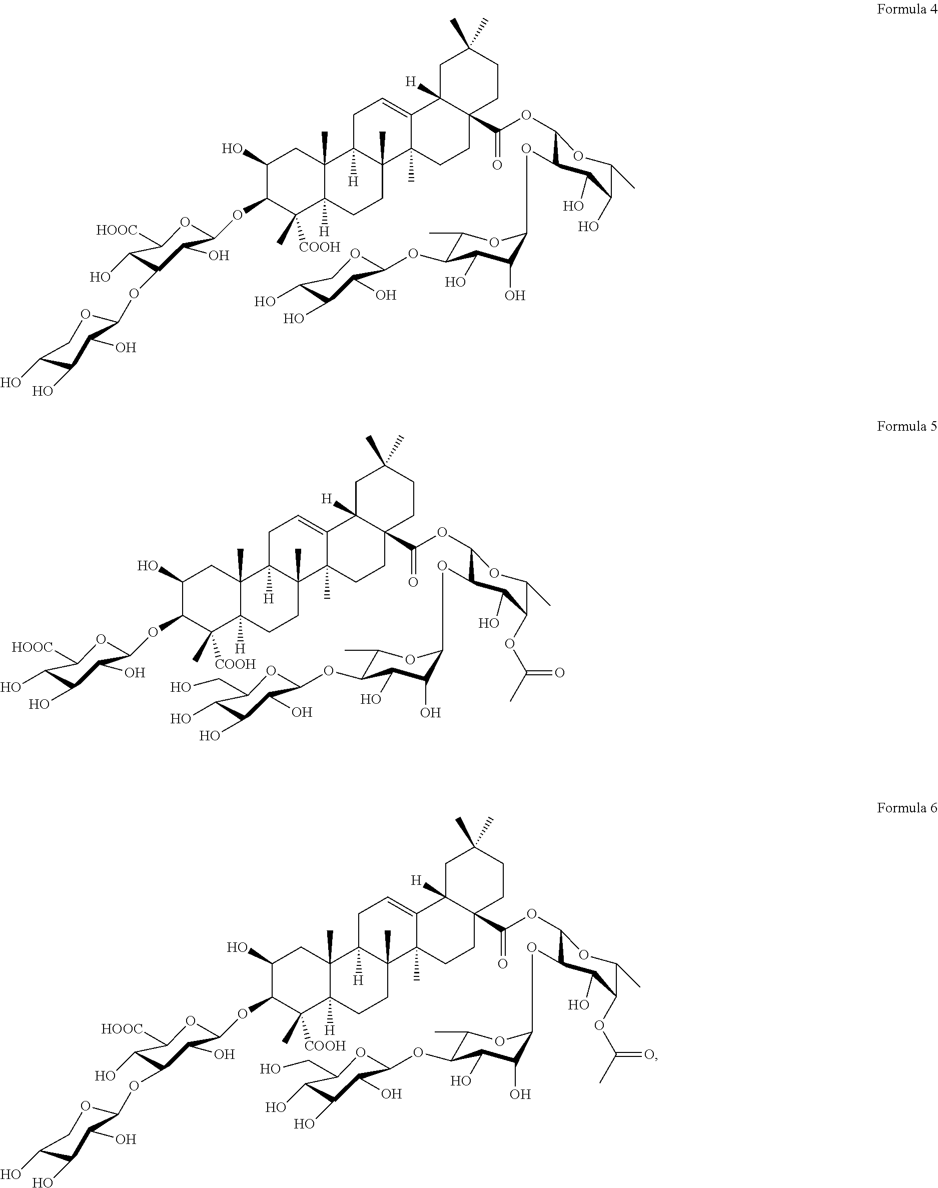

pharmaceutically acceptaable salts thereof, isomers thereof, hydrates thereof, and solvates thereof.

According to one embodiment of the present invention, it was confirmed that the novel glycosides of the above-described formulae 1 to 6 reduced the secretion of inflammatory cytokines in macrophages stimulated with LPS and effectively inhibited MUC5AC secretion in respiratory epithelial cells stimulated by PMA, thereby showing a preventive or therapeutic effect on inflammatory diseases, more specifically, inflammatory respiratory diseases. In addition, it was confirmed that the effects of these compounds were significantly superior, as compared to medicagenic acid.

In the present invention, the inflammatory disease may be selected from the group consisting of inflammatory respiratory disease, dermatitis, atopic dermatitis, allergy, psoriasis, bronchitis, ulcerative colitis, retinitis, uveitis, conjunctivitis, arthritis, rheumatoid arthritis, ankylosing spondylitis, osteoarthritis, nephritis, nephritis, autoimmune pancreatitis, chronic pelvic inflammatory disease, endometritis, otitis media, cystitis, and chronic prostatitis. Preferably, it is an inflammatory respiratory disease. However, the present invention is not limited thereto.

In the present invention, the inflammatory respiratory disease may be selected from the group consisting of asthma, pneumonia, acute lung injury, acute respiratory failure syndrome, chronic obstructive pulmonary disease, allergic rhinitis, bronchitis, pharyngitis, laryngitis, pharyngitis, and tonsillitis. However, the present invention is not limited thereto.

The pharmaceutical composition of the present invention may be formulated in various ways depending on the route of administration by methods known in the art along with one or more pharmaceutically acceptable carriers selected from the group consisting of compounds represented by the above-described formulae 1 to 6, pharmaceutically acceptable salts thereof, isomers thereof, hydrates thereof, and solvates thereof. The term “pharmaceutically acceptable” refers to non-toxic substances that are physiologically acceptable and do not interfere with the action of the active ingredient and do not usually cause gastrointestinal upset, allergic reactions such as dizziness, or similar reactions, when administered to human beings. The carriers comprise all kinds of solvents, dispersion media, oil-in-water or water-in-oil emulsions, aqueous compositions, liposomes, microbeads and microsomes.

The route of administration may be oral or parenteral. Parenteral administration may comprise, but are not limited to, intravenous, intramuscular, intraarterial, intramedullary, intrathecal, intracardiac, transdermal, subcutaneous, intraperitoneal, intranasal, enteral, topical, sublingual, or rectal administration.

When administered orally, the pharmaceutical composition of the present invention may be formulated into the forms of powder, granule, tablet, Pills, sugar-coated tablets, capsules, liquids, gels, syrups, suspensions, wafers, etc., according to methods known in the art, along with suitable carriers for oral administration. Examples of suitable carriers may comprise sugars such as lactose, dextrose, sucrose, sorbitol, mannitol, xylitol, erythritol and maltitol, and starches such as corn starches, wheat starches, rice starches, and potato starches, celluloses, fillers. In some cases, cross-linked polyvinylpyrrolidone, agar, alginic acid or sodium alginate may be added as disintegrants. Furthermore, the pharmaceutical composition may further comprise anti-coagulants, lubricants, wetting agents, fragrances, emulsifiers, and preservatives.

In addition, when administered parenterally, the pharmaceutical composition of the present invention may be formulated in the forms of injections, transdermal administration, and nasal inhalation according to methods known in the art, along with suitable parenteral carriers. The injections must be sterilized and protected from contamination by microorganisms such as bacteria and fungi. Examples of suitable carriers of the injections may comprise, but are not limited to, solvents or dispersion media comprising water, ethanol, polyols (e.g., glycerol, propylene glycol and liquid polyethylene glycol), mixtures thereof and/or vegetable oils. More preferably, suitable carriers may comprise Hanks' solution, Ringer's solution, phosphate buffered saline (PBS) containing triethanolamine, or isotonic solutions such as sterile water for injection, 10% ethanol, 40% propylene glycol, and 5% dextrose. In order to protect the injection from microbial contamination, various antibacterial and antifungal agents such as parabens, chlorobutanol, phenol, sorbic acid and thimerosal may be added to the injection. In addition, in most cases, the injection may additionally comprise an isotonic agent such as sugar or sodium chloride.

Transdermal administration agents have the forms of

ointments, creams, lotions, gels, topical solutions, paste preparations, linement preparations, aerol preparations, etc. In the above, the term “transdermal administration” means that a pharmaceutical composition is topically administered to skin so that an effective amount of the active ingredient contained in the pharmaceutical composition is delivered into the skin. For example, the pharmaceutical composition of the present invention may be prepared in an injectable formulation and administered by lightly pricking the skin with a 30-gauge thin injection needle or applying same directly to the skin. These formulations are described in formulas generally known in pharmaceutical chemistry.

For inhalation administration, the compounds used according to the invention may conveniently be delivered in the form of an aerosol spray from a pressurized pack or nebulizer, by using a suitable propellant (e.g., dichlorofluoromethane, trichlorofluoromethane, dichlorotetrafluoroethane, carbon dioxide and other suitable gases). For pressurized aerosols, the dosage unit may be determined by providing a valve that delivers a metered amount. For example, gelatin capsules and cartridges for use in inhalers or insufflators may be formulated to contain a compound and a powder mixture of suitable powder bases such as lactose or starch.

For other pharmaceutically acceptable carriers, those known in the art may be referred to.

In addition, the pharmaceutical composition according to the present invention may further comprise one or more selected from the group consisting of buffers (e.g., saline and PBS), carbohydrates (e.g., glucose, mannose, sucrose and dextran), antioxidants, bacteriostatic agents, chelating agents (e.g., EDTA and glutathione), adjuvants (e.g., aluminum hydroxide), suspending agents, thickening agents and/or preservatives.

In addition, the pharmaceutical compositions of the present invention may be formulated by using methods known in the art to provide rapid, sustained or delayed release of the active ingredient after administration to a mammal.

In addition, the pharmaceutical composition of the present invention may be administered in combination with known substances that are effective in preventing or treating inflammatory diseases.

In the present invention, the amount of the composition is not greatly limited depending on the purpose or aspect of use. For example, it may be 0.01 to 99% by weight, preferably 0.5 to 50% by weight, more preferably 1 to 30% by weight, based on the total weight of the composition. In addition, the pharmaceutical composition according to the present invention may further comprise additives such as pharmaceutically acceptable carriers, excipients, or diluents, in addition to the active ingredients. The pharmaceutical composition of the present invention may comprise 0.1 to 99.9% by weight of the compounds represented by formulae 1 to 6 prepared by the method of the present invention, and 99.9% to 0.1% by weight of the carriers.

The pharmaceutical composition according to the present invention may comprise a pharmaceutically effective amount of the above-described compound alone or may additionally comprise one or more pharmaceutically acceptable carriers. The pharmaceutical composition of the present invention may be administered to a patient in a single dose, or may be administered by a fractionated treatment protocol in which multiple doses are administered over a long period of time. In the above, the pharmaceutically effective amount refers to an amount that shows a greater response, as compared to the negative control, and, preferably, refers to an amount sufficient to treat or prevent inflammatory diseases. The effective amount of the strain according to the present invention may be 0.001 to 1000 mg/kg b.w./day, preferably 1 to 2000 mg/kg b.w./day. However, the present invention is not limited thereto. However, the pharmaceutically effective amount may vary appropriately depending on various factors such as disease and its severity, patient's age, weight, health status, gender, route of administration and treatment period.

The present invention also provides a pharmaceutical composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

The present invention also provides a pharmaceutical composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 30 to 100% (v/v) of a non-polar solvent aqueous solution in step (b).

The present invention also provides a pharmaceutical composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 30 to 100% (v/v) of a methanol aqueous solution in step (b).

In the present invention, the fractions may be characterized in that they comprise one or more compounds selected from the group consisting of formulae 1 to 6.

For detailed description of steps (a) and (b) in the

present invention, the above description may be referred to.

The present invention also provides a food composition for preventing or improving inflammatory diseases comprising as an active ingredient one or more selected from the group consisting of the compounds represented by the following formulae 1 to 6:

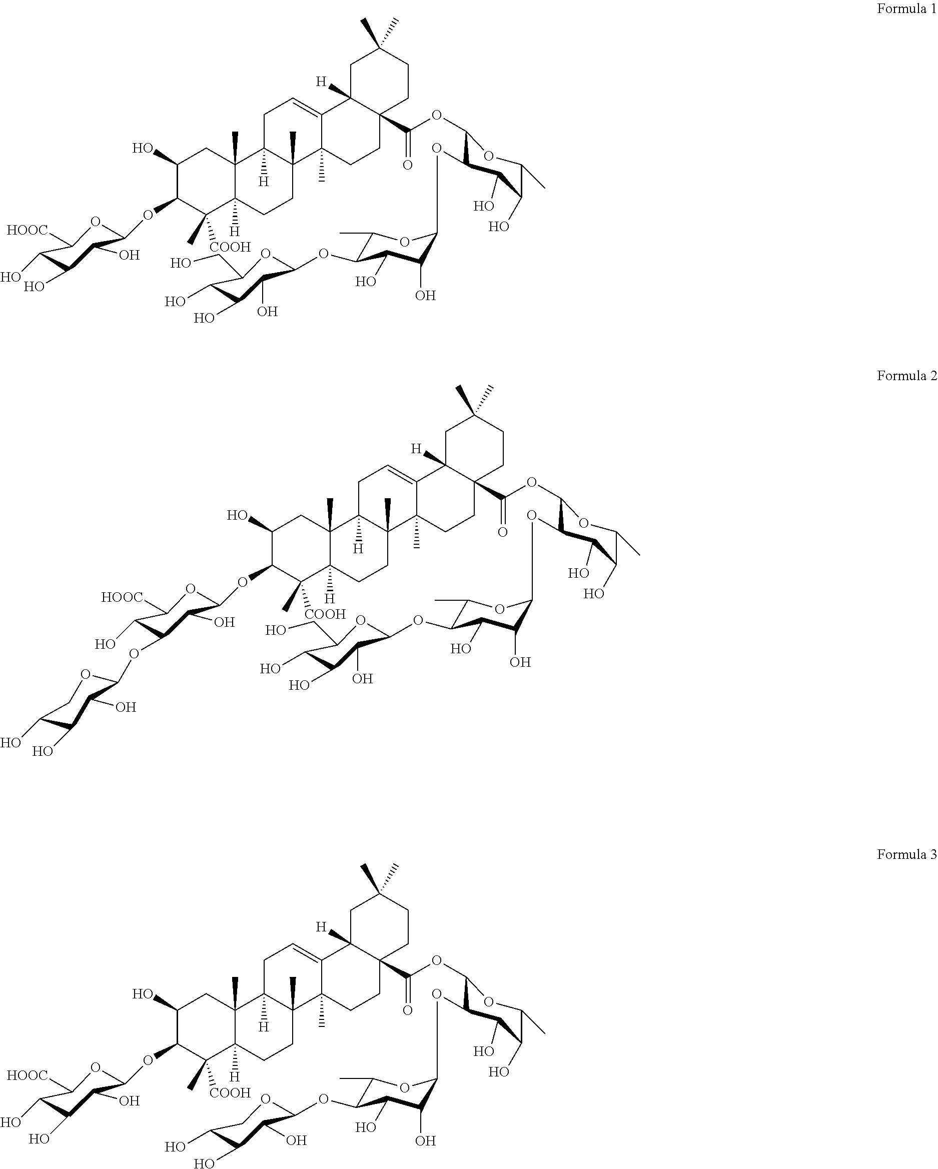

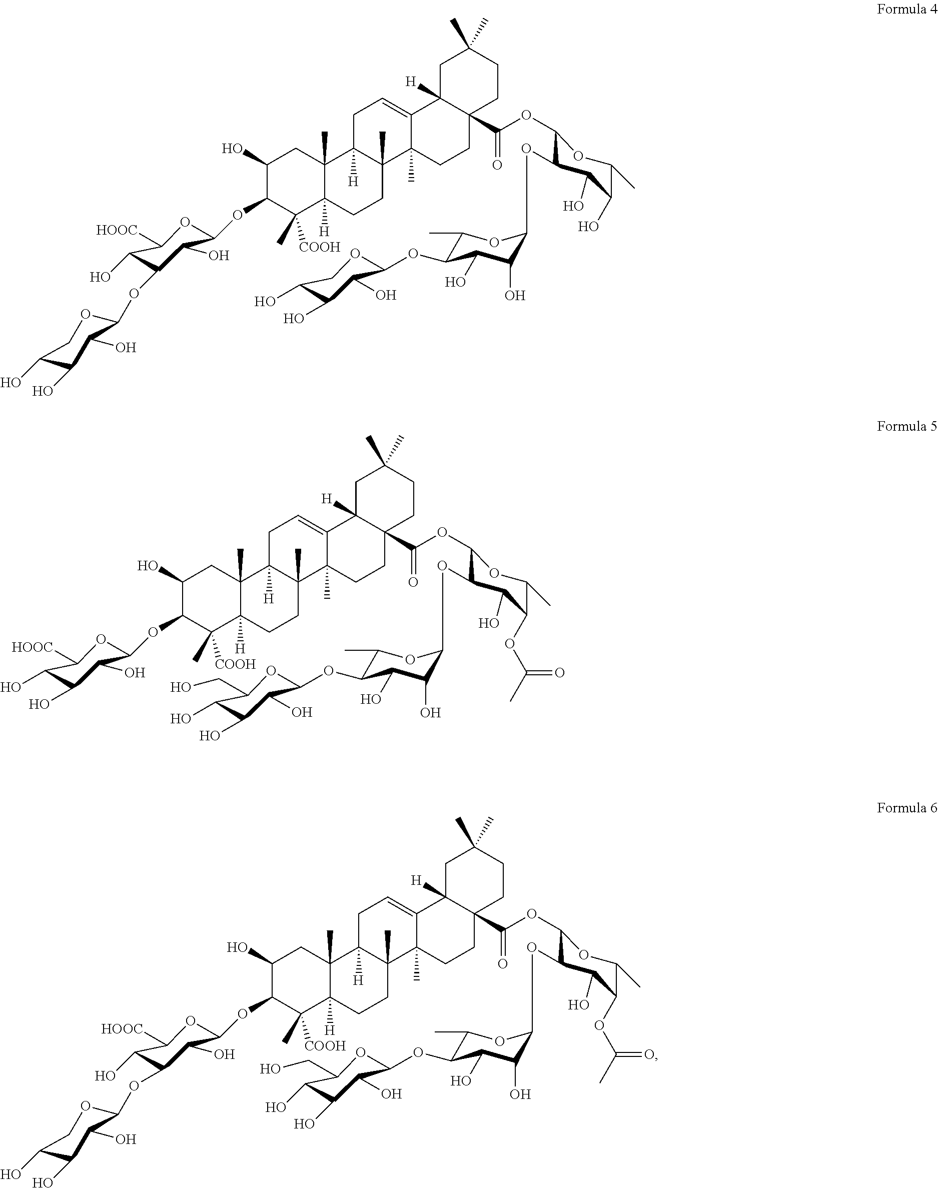

pharmaceutically acceptable salts thereof, isomers thereof, hydrates thereof, and solvates thereof.

The food composition of the present invention has all types of functional foods, nutritional supplements, health foods, health functional foods, and food additives. These types of food compositions may be prepared in various forms according to conventional methods known in the art.

For example, health foods may be prepared and consumed in the form of tea, juice, and drinks comprising the compounds selected from the above-described formulae 1 to 6, or may be consumed by granulating, encapsulating, or powdering them. In addition, compounds selected from the above-described formulae 1 to 6 of the present invention may be prepared in the form of a composition by mixing them with known substances or active ingredients known to be effective against inflammatory diseases. For example, the food composition of the present invention may further comprise trace amounts of minerals, vitamins, lipids, sugars, and components known to produce effects of preventing or treating inflammatory diseases, in addition to the compounds selected from the above-described formulae 1 to 6. The minerals may comprise nutrients necessary for growth, such as calcium and iron, and the vitamins may comprise vitamin C, vitamin E, vitamin B1, vitamin B2, and vitamin B6. Lipids may comprise alkoxyglycerol or lecithin, and sugars may comprise fructooligosaccharides.

In addition, functional foods may be prepared by adding the composition of the present invention to beverages (e.g., alcoholic beverages), fruit and its processed foods (e.g., canned fruit, bottled food, jam and marmalade), fish, meat and its processed foods (e.g., ham and sausage corn beef), bread and noodles (e.g., udon, buckwheat noodles, ramen, spaghetti and macaroni), fruit juice, various drinks, cookies, taffy, dairy products (e.g., butter and cheese), edible vegetable oil, margarine, vegetable protein, retort food, frozen food, various seasonings (e.g., soybean paste, soy sauce and sauce), and the like.

The present invention also provides a food composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 20 to 100% (v/v) of a non-polar solvent aqueous solution in step (b).

The present invention also provides a food composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 30 to 100% (v/v) of a non-polar solvent aqueous solution in step (b).

The present invention also provides a food composition for preventing or treating inflammatory diseases, wherein the composition is prepared by a method comprising (a) extracting spinach with a solvent selected from the group consisting of water, organic solvents, subcritical fluids, supercritical fluids and mixtures thereof; and (b) fractionating the spinach extract by chromatography according to a concentration gradient by using water and a non-polar solvent as mobile phases, and the composition comprises as an active ingredient fractions fractionated from 30 to 100% (v/v) of a methanol aqueous solution in step (b).

For detailed description of steps (a) and (b) in the present invention, the above description may be referred to.

The present invention also provides a veterinary composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

The veterinary composition of the present invention may further comprise appropriate excipients and diluents according to conventional methods. The excipients and diluents may comprise lactose, dextrose, sucrose, sorbitol, mannitol, xylitol, erythritol, maltitol, starch, gum acacia, alginate, gelatin, calcium phosphate, calcium silicate, cellulose, methyl, cellulose, microcrystalline cellulose, polyvinyl pyrrolidone, water, methylhydroxybenzoate, propylhydroxybenzoate, talc, magnesium stearate, cetanol, stearyl alcohol, liquid paraffin, sorbitan monostearate, polysorbate 60, methylparaben, propylparaben and mineral oil.

The veterinary composition according to the present invention may further comprise fillers, anti-coagulants, lubricants, wetting agents, spices, emulsifiers, preservatives, etc. The veterinary composition of the present invention may be formulated by using methods known in the art to provide rapid, sustained or delayed release of the active ingredient after administration to an animal. The formulation may be in the form of powders, granules, tablets, capsules, suspensions, emulsions, solutions, syrups, aerosols, soft or hard gelatin capsules, suppositories, sterile injectable solutions, sterile external preparations, etc.

The veterinary composition according to the present invention may vary depending on the age, sex, and weight of the animal, but may be administered in an amount of 0.1 to 100 mg/kg once or several times a day, and the dosage may be increased or decreased depending on the route of administration, severity of disease, gender, weight, age. Accordingly, the above-described dosage does not limit the scope of the present invention in any way.

The present invention also provides a feed composition for preventing or improving inflammatory diseases comprising as an active ingredient one or more selected from the group consisting of the compounds represented by the above-described formulae 1 to 6, pharmaceutically acceptable salts thereof, isomers thereof, hydrates thereof, and solvates thereof.

The ‘feed composition’ according to the present invention may comprise, as active ingredients, food raw materials usable as food listed in the Food Standards and Specifications ('Food Code') and food additives described in the Food Additives Code, in addition to the above-described components. Even if they are not food raw materials usable as food or food additives, the feed composition may comprise raw materials that fall within the scope of sweet feed in Annex Table 1 of ‘Standards and Specifications for Feed, etc.’ and raw materials that fall within the scope of supplementary feed in Annex Table 2.

The ‘feed composition’ may be an extractant among supplementary feeds according to ‘Standards and specifications for feed, etc.’, or may be a compound feed comprising the supplementary feed.

When preparing the feed composition, the amount of the compounds represented by the above-described formulae 1 to 6 does not need to be specifically limited as long as it prevents or improves inflammatory diseases. However, they may be contained, for example, 0.1 to 99% by weight, 0. 5 to 95% by weight, 1 to 90% by weight, 2 to 80% by weight, 3 to 70% by weight, 4 to 60% by weight, or 5 to 50% by weight.

Specifically, the amount of the compounds represented by the above-described formulae 1 to 6 may be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2 .5, 2.6, 2.7, 2.8, 2.9, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 73, 74 , 75, 76, 77, 78, 79, 80. 81, 82, 83, 84, 85, 86, 87, 88, 89, 90, 91, 92, 93, 94, 95, 96, 97, 98, or 99% by weight.

The compounds of the above-described formulae, which are an active ingredient in the feed composition, vary depending on the condition and weight of the animal consuming same, and presence, degree and duration of disease, but may be appropriately selected by those skilled in the art. For example, the daily dosage may be 1 to 5,000 mg, preferably 5 to 2,000 mg, more preferably 10 to 1,000 mg, even more preferably 20 to 800 mg, most preferably 50 to 500 mg. In addition, the number of administrations does not need to be particularly limited, but may be adjusted by those skilled in the art within the range of 3 times a day to once a week. In the case of long-term intake for health and hygiene purposes or health control, the daily dosage and number of administrations may be below the above-described ranges.

The present invention provides use of one or more selected from the group consisting of the compounds represented by the above-described formulae 1 to 6, pharmaceutically acceptable salts thereof, isomers thereof, hydrates thereof, and solvates thereof, for preparing a composition for treating inflammatory diseases.

The present invention provides a method of treating inflammatory diseases comprising administering to a subject in need thereof an effective amount of a composition comprising as an active ingredient one or more selected from the group consisting of the compounds represented by the above-described formulae 1 to 6, pharmaceutically acceptable salts thereof, isomers thereof, hydrates thereof, and solvates thereof.

The term “effective amount” of the present invention refers to an amount that exhibits an effect of improving, treating, detecting, diagnosing of a wound, or inhibiting or reducing the progression of a wound, when administered to a subject. In addition, the term “individual” may be an animal, preferably a mammal, particularly an animal comprising a human being, and may also be a cell, tissue, or organ derived from an animal. The subject may be a patient in need of the effect.

The term “treatment” of the present invention comprehensively refers to improving wounded area or a symptom caused by a wound, may comprise curing, substantially preventing, or improving the condition of the wound, and comprises mitigating, curing, or preventing one or most of the symptoms caused by the disease, but the present invention is not limited thereto.

As used herein, the term “comprising” is used in the same sense as “including” or “characterized by.” The composition or method according to the present invention does not exclude additional components or method steps not specifically mentioned. In addition, the term “consisting of” refers to excluding additional elements, steps or components not separately described. The term “essentially consisting of” means that, in addition to the described materials or steps, materials or steps that do not substantially affect the basic characteristics thereof may be contained in the scope of a composition or method.

### Advantageous Effects Of Invention

The novel glycoside compound and the spinach extract fraction comprising same provided by the present invention are effective for effectively inhibiting the secretion of excessive inflammatory cytokines from immune cells and suppressing excessive secretion of mucus in inflammatory respiratory diseases such that they may be used in preventing or treating various inflammatory diseases such as inflammatory respiratory diseases.

### BRIEF DESCRIPTION OF DRAWINGS

FIG. 1 is a diagram showing the results of UPLC analysis of a spinach extract.

FIG. 2 shows the preparation process of an effective fraction (Fr.2) comprising five (5) novel substances and celosin I compound from a spinach extract.

FIGS. 3A and 3B are diagrams showing the results of CAD chromatogram analysis of a spinach extract, fraction 1 (Fr.1) and fraction 2 (Fr.2).

FIGS. 4A and 4B show the results of UPLC-QTOF-MS analysis of five (5) novel substances (i.e., SOA1, SOA2, SOA4, SOA5 and SOA6) and celosin I compound (i.e., SOA 3) isolated from a spinach extract.

FIGS. 5A and 5B show the results of evaluating the cytotoxicity (5A) and the ability to inhibit the secretion of TNF-alpha (5B) by a spinach extract (SO_total), its butanol fraction (SO_BuOH), fraction 1 (SO_Fr.1) and fraction 2 (SO_Fr.2) in macrophages stimulated with LPS.

FIG. 6 is a table showing the results of evaluating the cytotoxicity and the ability to inhibit the secretion of TNF-alpha and NO by five (5) novel substances isolated from a spinach extract (i.e., SOA1, SOA2, SOA4, SOA5, SOA6), celosin I compound (SOA 3) and medicagenic acid (MA) in macrophages stimulated with LPS.

FIGS. 7A to 7C show the results of evaluating the ability to inhibit the secretion of NO and PGE2 (7A, 7B) and the ability to inhibit the expression of COX-2 and iNOS protein (7C) by Fraction 2 of a spinach extract (SO_Fr.2) in LPS-stimulated macrophages, according to treatment with different concentrations.

FIGS. 8A to 8C show the results of evaluating the ability to inhibit the secretion of inflammatory cytokines IL-4 (9A), IL-5 (9B) and IL-13 (9C) by fraction 2 (SO_Fr.2) of a spinach extract in EL4 cells stimulated with PMA and lonomycin.

FIGS. 9A and 9B show the results of evaluating the cytotoxicity (9A) and the ability to inhibit the secretion of mucus (9B) by a spinach extract (SO_total), its butanol fraction (SO_BuOH), fraction 1 (SO_Fr.1) and fraction 2 (SO_Fr.2) in PMA-stimulated respiratory epithelial cells.

FIGS. 10A and 10B show the results of evaluating the cytotoxicity 10A) and the ability to inhibit the secretion of mucus (10B) by five (5) novel substances isolated from a spinach extract (i.e., SOA1, SOA2, SOA4, SOA5, SOA6) and celosin I compound (SOA 3) in respiratory epithelial cells stimulated with PMA.

FIGS. 11A to 11D show the results of evaluating the ROS (11A), elastase (11B), TNF-alpha (11C) and IL-6 (11D) secretion inhibitory ability of fraction 2 of a spinach extract in bronchoalveolar lavage fluid after administered at the indicated concentrations (S5: 5 mg/kg, S10: 10 mg/kg) in mouse models of chronic obstructive pulmonary disease (COPD).

FIGS. 12A to 12D show the results of evaluating the IL-4 (12A), IL-5 (12B), IL-13 (12C) and IgE (12D) inhibitory ability of fraction 2 of a spinach extract in bronchoalveolar lavage fluid after administered at the indicated concentrations (S5: 5 mg/kg, S10: 10 mg/kg) in asthma animal models.

FIG. 13 shows the results of measuring the number of inflammatory cells in bronchoalveolar lavage fluid by fraction 2 of a spinach extract after administered at the indicated concentrations (S5: 5 mg/kg, S10: 10 mg/kg) in pneumonia mouse models.

FIGS. 14A to 14C show the results of evaluating the ability to inhibit the expression of ROS (14A), TNF-alpha (14B) and IL-6 (14C) in bronchoalveolar lavage fluid by fraction 2 of a spinach extract after administered at the indicated concentrations (S5: 5 mg/kg, S10: 10 mg/kg) in pneumonia mouse models.

### MODE FOR INVENTION

Hereinafter, the present invention will be described in detail by the following embodiments. However, the following embodiments are only for illustrating the present invention, and the present invention is not limited thereto.

### Example 1

### Preparation of a Spinach Extract and Analysis of its Component Profile

100 kg of spinach was extracted at 50° C. for 90 minutes by using 18 times 30% alcohol, and then filtered/concentrated.

The components of the prepared spinach extract were analyzed by UPLC.

Specifically, for UPLC analysis, the spinach extract was filtered once through a 0.25 mm membrane filter for UPLC. A UPLC instrument (Waters UPLC-Q-TOF) was equipped with a column (Waters BEH C18 column, 2.1×100 mm, 1.7 μm), and each filtered fraction was loaded in an amount of 0.3 μl.

At this time, the solvent used for UPLC analysis was acetonitrile+0.1% formic acid/water+0.1% formic acid 10:90->100:0 (v/v), and the elution rate was 0.4 ml/min.

The separation degree of materials separated from UPLC was confirmed in chromatographic format by using Mass spectrometry (MS) and Charged Aerosol Detector (CAD) as detectors.

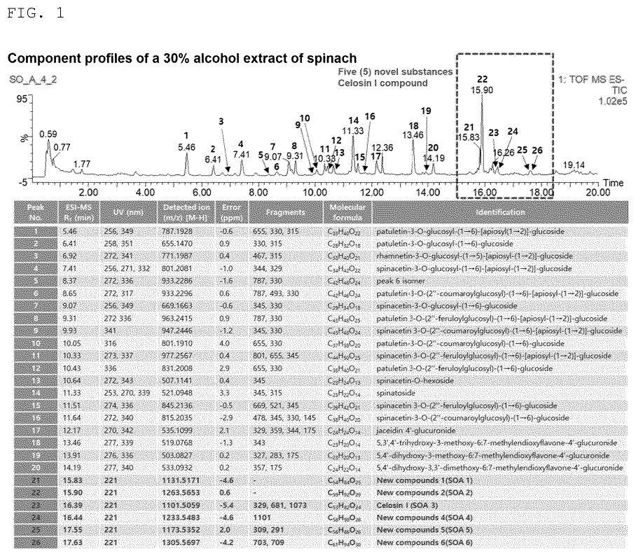

The UPLC-QTOF-MS analysis conditions and results are shown in FIG. 1 and Table 1.

TABLE 1

| Time(min) | Flow(ml/min) | % A | % B |
|---|---|---|---|
| 0.00 | 0.400 | 90 | 10 |
| 1.00 | 0.400 | 90 | 10 |
| 7.00 | 0.400 | 85 | 15 |
| 13.00 | 0.400 | 70 | 30 |
| 16.00 | 0.400 | 60 | 40 |
| 23.00 | 0.400 | 20 | 80 |
| 25.00 | 0.400 | 0 | 100 |
| 28.00 | 0.400 | 0 | 100 |
| 28.30 | 0.400 | 90 | 10 |
| 30.00 | 0.400 | 90 | 10 |

As can be seen in FIG. 1, five (5) novel substances (i.e., SOA1, SOA2, SOA4, SOA5, SOA6) and celosin I compound (SOA 3) were identified from the spinach extract through UPLC analysis.

### Example 2

### Fractionation of a Spinach Alcohol Extract

Effective fractions and novel compounds were isolated from the extract obtained in Example 1 by the following method.

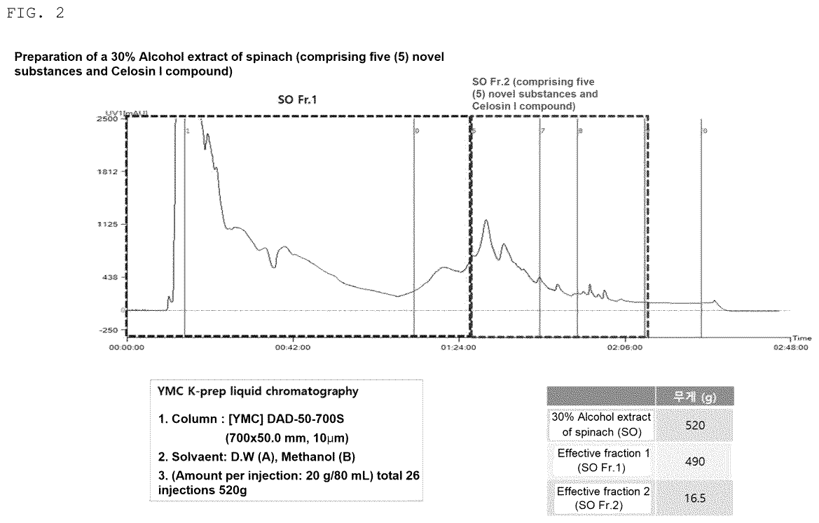

Specifically, an MPLC instrument (YMC LAB-300) was equipped with a column (YMC-DAD-50-700S (50×700 mm, 10 μm), and a spinach extract (20 g) was loaded. At this time, the solvent was methanol/water 10:90->100:0 (v/v), and the elution rate was 100 ml/min, and detection at the wavelengths of UV 210, 254, 280 nm was conducted to obtain small fractions (i.e., SO Fr.1 and Fr.2) (FIG. 2).

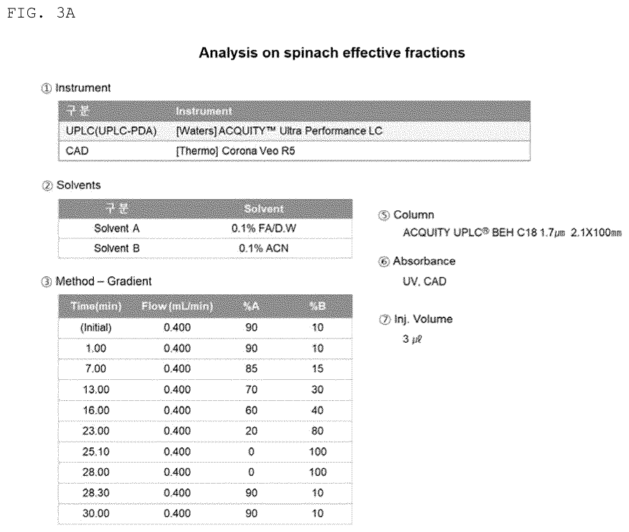

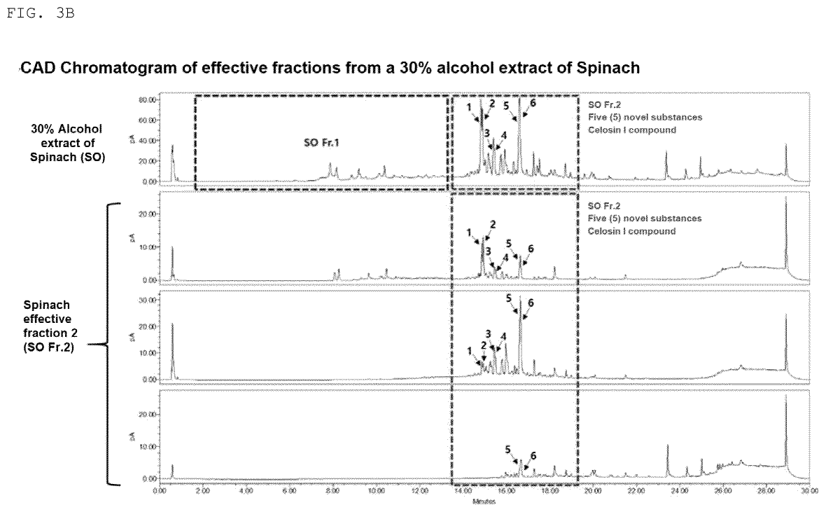

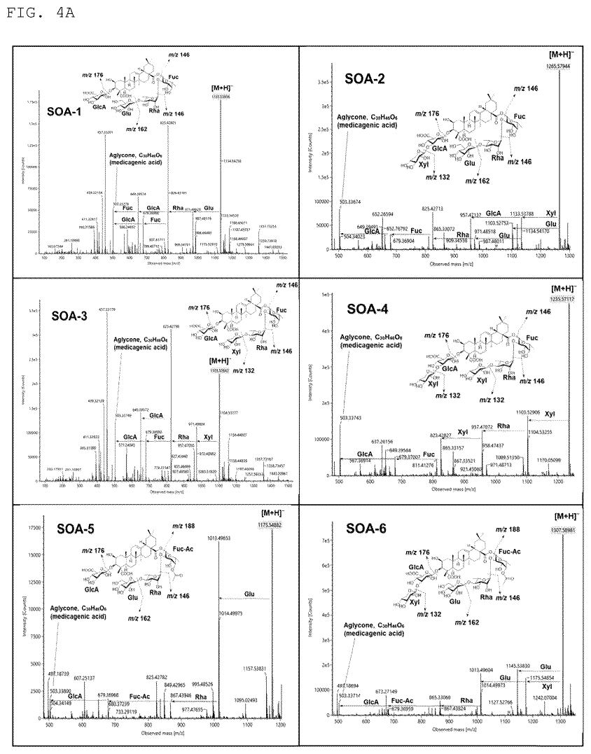

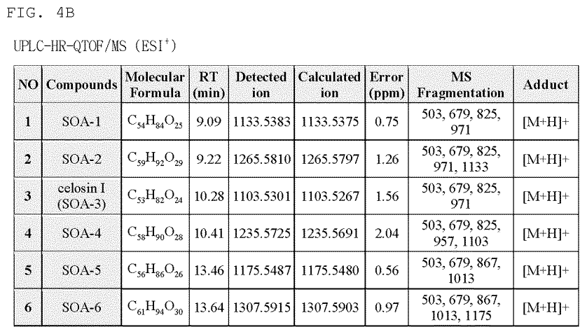

Specifically, an MPLC instrument (YMC Lc-Forte/R) was equipped with a column (Waters X-bridge, 19×250 mm, 5 μm), and effective fraction SO Fr.2 (16.5 g) was loaded. At this time, the solvent was acetonitrile+0.1% formic acid/water+0.1% formic acid 15:85->100:0 (v/v), and the elution rate was 11 ml/min. Detection was conducted at UV wavelengths of 210, 254, and 280 nm, and compounds SO peak 1 (SOA1, 238.6 mg), 2 (SOA2, 170.2 mg), 3 (SOA3, 39.4 mg) and 4 (SOA4, 24.2 mg) with new structures represented by formulae 1, 2, 3, and 4 having the following physical properties were obtained (FIGS. 3A, 3B, 4A and 4B).

### Example 3

### Structural Analysis of Five (5) Novel Substances

The molecular weight and molecular formula of the oleanan-type triterpene saponin compound obtained in Example 2 were determined by using a high-resolution Qtof-MS mass spectrometer (Vion IMS-Qtof-MS (Waters, USA)). In addition, the molecular structure was determined by using spectroscopic data ¹H-NMR, ¹³C-NMR and 2D NMR (COZY, HSQC, HMBC, ROESY) through nuclear magnetic resonance (NMR) analysis (Bruker Avance-800 MHz, Bruker Avance-900 MHz Bruker, Germany).

As a result of comparing the instrumental analysis results with those of published literature, the oleanan type triterpene saponin represented by the following formulae 1 to 6 was identified. The specific analysis results are as follows.

In Compound 1, the molecular ion M+H⁺ of m/z 1133.5383 was observed in the HR-Qtof-MS spectrum, and, through this, the molecular formula of C₅₄H₈₄O₂₅ was determined. Through MS cleavage pattern analysis, a cleavage ion at m/z 971 due to loss of glucose (162 Da), and molecular ions of m/z 825 and 679 due to loss of the pentose sugars rhamnose and fucose (146 Da) were observed. In addition, the molecular ion of sapogenin m/z 503 due to the loss of glucuronic acid (176 Da) was detected, which is the same as the molecular ion value of medicagenic acid. In the ¹H NMR spectrum, eight methyl protons (i.e., δ_{H} 1.89, 1.64, 1.45, 1.40, 1.19, 1.03, 0.79 and 0.77), one olefinic proton (i.e., δ_{H} 5.33), four anomeric protons (i.e., δ_{H} 6.20, 5.91, 5.13 and 5.06), and a methine proton with two adjacent oxygens (i.e., δ_{H} 4.74 and 4.63) were observed. In the ¹³C NMR spectrum, three carbonyl carbons (i.e., δ_{C} 180. 9, 176. 6 and 172. 7), a pair of olefinic carbons (i.e., δ_{C} 143.8 and 122.4), four enomeric carbons (i.e., δ_{C} 106.2, 105.2, 101.1 and 94.5), and methylene carbon substituted with one oxygen (i.e., δ_{C} 62.4), and 18 methine carbons (i.e., δ_{C} 85.7, 84.5, 78.1, 77.9, 77.2, 76.9, 75.9, 75.8, 74.4, 74.1, 72.8, 72.6, 72.1, 71.9, 71.3, 71.2, 70.0 and 68.2) were confirmed. As a result of confirmation through 2D NMR, a signal that the methyl proton peak δ_{H} 1.89 (3H, s, H3-24) was adjacent to the carbonyl peak (δ_{C} 180. 9) was observed. In addition, through this NMR spectrum pattern and existing prior literature (J. Agric. Food Chem., 2005, 53, 2164-2170, J. Asian Nat. Prod. Res., 2014, 16, 240-247, Phytochemistry 2020, 169, 112162), it was confirmed that Compound 1 is an oleananic saponin compound with medicagenic acid (2β,3β-dihydroxyolean-12-ene-23,28-dioic acid) as an aglycone. In addition, 2D NMR (COZY, HSQC and HMBC) confirmed that among the four enomeric protons, the peak at δ_{H} 5.13 (1H, d, J=8.1 Hz, , H-1′, GlcA) was connected to the peak at δ_{C} 85.7 (C-3), and the peak at δ_{H} 5.91 (1H, d, J)=8.0 Hz, H-1″, Fuc) was connected to the carbonyl group at δ_{C} 176.6 (C-28). It was also confirmed that the peak at δ_{H} 6.20 (1H, d, J=1.8 Hz, H-1″′, Rham) was connected to carbon at position 2 of fucose (δ_{C} 74.1, C-2″), and the peak at δ_{H} 5.06 (1H, d, J=7.2 Hz, H-1″″, Glu) was bound to the carbon at position 4 of rhamnose (δ_{C} 84.5 C-4″′). Combining previous literature and the above-described results, Compound 1 was identified as 3-O-β-D-glucuronopyranosyl-2β,3β-dihydroxyolean-12-ene-23,28-dioic acid-28-O-β-D-glucopyranosyl-(1→4)-α-L-rhamnopyranosyl-(1→2)-β-D-fucopyranoside, and was named spinasaponin C.

In Compound 2, the molecular ion M+H⁺ of m/z 1265.5810 was observed in the HR-Qtof-MS spectrum, and, through this, the molecular formula of C₅₉H₉₂O₂₉ was determined. Through MS cleavage pattern analysis, a molecular ion of m/z 1133 due to loss of xylose (132 Da), a pentose sugar, was detected. In addition, a cleavage ion at m/z 971 due to loss of glucose (162 Da) and molecular ions at m/z 825 and 679 due to loss of pentose sugars rhamnose and fucose (146 Da) were observed. In addition, m/z 503, the molecular ion of sapogenin due to the loss of glucuronic acid (176 Da) was detected, and the molecular ion value was identical to that of medicagenic acid. As a result of comparative analysis of the 1D and 2D NMR spectra and the prior literature, Compound 2 showed a spectrum similar to Compound 1, and it was confirmed that xylose sugar, a pentose sugar, was additionally connected to Compound 1. 2D NMR data confirmed that the enomeric proton of the xylose sugar (δ_{H} 5.30, 1H, d, J=7.2 Hz, H-1″, Xyl) was connected to C-3′ of glucuronic acid (δ_{C} 86.1, C-3′, GlcA). Therefore, combining the prior literature and the above-described results, Compound 2 was identified as 3-O-≢-D-xylopyranosyl-(1→3)-β-D-glucuronopyranosyl-2β,3β-dihydroxyolean-12-ene-23, 28-dioic acid-28-O-β-D-glucopyranosyl-(1→4)-α-L-rhamnopyranosyl-(1→2)-β-D-fucopyranoside, and was named spinasaponin D.

In Compound 3, the molecular ion M+H⁺ of m/z 1103.5301 was observed in the HR-Qtof-MS spectrum, and, through this, the molecular formula of C₅₃H₈₂O₂₄ was determined. Through MS cleavage pattern analysis, a molecular ion of m/z 971 due to loss of xylose (132 Da), a pentose sugar, was detected. Molecular ions of m/z 825 and 679 were observed due to the loss (146 Da) of the pentose sugars rhamnose and fucose. In addition, m/z 503, the molecular ion of sapogenin due to the loss of glucuronic acid (176 Da) was detected, and the molecular ion value was identical to that of medicagenic acid. As a result of comparative analysis of 1D and 2D NMR spectra and the prior literature, Compound 3 showed a spectrum similar to Compound 1. In addition, it was confirmed that the position of the glucose sugar in Compound 1 was replaced with xylose sugar, a pentose sugar, and connected to Compound 1. 2D NMR data confirmed that the enomeric proton of xylose sugar (δ_{H} 5.03, 1H, d, J=7.2 Hz, H-1″″, Xyl) was connected to C-4″′ of rhamnose (δ_{C} 85.8, C-4″′, Rham). When combining the above-described results, it was confirmed that Compound 3 was the compound 3-O-β-D-glucuronopyranosyl-2β,3β,-dihydroxyolean-12-ene-23,28-dioic acid-28-O-β-d-xylopyranosyl-(1→4)-α-1-rhamnopyranosyl-(1→2)-β-D-fucopyranoside (Celosin I) reported in the prior literature (J. Asian Nat. Prod. Res., 2014, 16, 240-247).

In Compound 4, the molecular ion M+H⁺ of m/z 1235.5725 was observed in the HR-Qtof-MS spectrum, and, through this, the molecular formula of C58H90028 was determined. Through MS cleavage pattern analysis, a molecular ion of m/z 1103 due to loss of xylose (132 Da), a pentose sugar, was detected. Molecular ions of m/z 957, 825 and 679 due to the loss of rhamnose (146 Da), xylose, and fucose (146 Da) were observed. In addition, m/z 503, the molecular ion of sapogenin, due to the loss of glucuronic acid (176 Da) was detected, the molecular ion value was identical to that of medicagenic acid. As a result of comparative analysis of the 1D and 2D NMR spectra and the prior literature, Compound 4 showed a spectrum similar to Compound 2, and it was confirmed that the glucose sugar of Compound 2 was substituted with xylose sugar, a pentose sugar, and was connected to Compound 2. 2D NMR data confirmed that an additional enomeric proton of the xylose sugar (δ_{H} 4.94, 1H, d, J=7.2 Hz, H-1″″′, Xyl-II) was connected to C-4″″ of rhamnose (δ_{C} 84.6, C-3″″, Rham). Therefore, by combining prior literature and the above-described results, Compound 4 was identified as 3-O-β-D-xylopyranosyl-(1→3)-β-D-glucuronopyranosyl-2β,3β-dihydroxyolean-12-ene-23,28-dioic acid-28-O-β-D-xylopyranosyl-(1→4)-α-L-rhamnopyranosyl-(1→2)-β-D-fucopyranoside, and was named spinasaponin E.

In Compound 5, the molecular ion M+H⁺ of m/z 1175.5487 was observed in the HR-Qtof-MS spectrum, and, through this, the molecular formula of C₅₆H₈₆O₂₆ was determined. Through MS cleavage pattern analysis, a cleavage ion of m/z 1013 due to loss of glucose (162 Da) and a molecular ion of m/z 867 due to loss of rhamnose, a pentose sugar (146 Da), were observed. In addition, the molecular ion of m/z 679 due to loss of acetylated fucose (188 Da) and the molecular ion of sapogenin, m/z 503, due to loss of glucuronic acid (176 Da) were detected, and the molecular ion value was identical to that of medicagen acid. As a result of comparative analysis of the 1D and 2D NMR spectra and the prior literature, Compound 5 showed a spectrum similar to Compound 1, and it was confirmed that Compound 5 had an acetyl group added to the skeleton of Compound 1 (δ_{H} 4.94 3H, s, OAc—CH3; δ_{C} 171.3, OAc, 20.6, OAc—CH3). 2D NMR data confirmed that the acetyl group was substituted at position 4 of the fucose sugar (δ_{C} 74.5, C-4″, Fuc), and the enomeric proton of the acetylated fucose sugar (δ_{H} 5.95, 1H, d, J=8.1 Hz, H-1″, Fuc) was connected to the carbonyl group of methikagenic (δ_{C} 176. 6, C-28). Therefore, by combining the prior literature and the above-described results, Compound 5 was identified as 3-O-β-D-glucuronopyranosyl-2β,3β-dihydroxyolean-12-ene-23,28-dioic acid-28-O-β-D-glucopyranosyl-(1→4)-α-L-rhamnopyranosyl-(1→2)-4-O-acetyl-β-D-fucopyranoside, and was named spinasaponin F.

In Compound 6, the molecular ion M+H⁺ of m/z 1307.5915 was observed in the HR-Qtof-MS spectrum, and, through this, the molecular formula of C61H94O30 was determined. Through MS cleavage pattern analysis, a molecular ion of m/z 1175 due to loss of xylose (132 Da), a pentose sugar, was detected, and a cleaved ion of m/z 1013 due to loss of glucose (162 Da) and a molecular ion of m/z 867 due to loss of rhamnose, a pentose sugar (146 Da), were observed, and a molecular ion of m/z 679 due to loss of acetylated fucose (188 Da) was detected. In addition, m/z 503, the molecular ion of sapogenin, due to the loss of glucuronic acid (176 Da) was detected, and the molecular ion value was identical to that of medicagenic acid. As a result of comparative analysis of the 1D and 2D NMR spectra and the prior literature, Compound 6 showed a spectrum similar to Compound 5, and it was confirmed that xylose sugar, a pentose sugar, was additionally connected to Compound 5. 2D NMR data confirmed that the enomeric proton of xylose sugar (δ_{H} 5.15, 1H, d, J=7.2 Hz, H-1″, ′, GlcA) was connected to C-3′ of glucuronic acid (δ_{C} 85.6, C-3′, GlcA). Therefore, by combining the prior literature and the above-described results, Compound 6 was identified as 3-O-β-D-xylopyranosyl-(1→3)-β-D-glucuronopyranosyl-2β,3β-dihydroxyolean-12-ene-23, 28-dioic acid-28-O-β-D-glucopyranosyl-(1→4)-α-L-rhamnopyranosyl-(1→2)-4-O-acetyl-β-D-fucopyranoside, and was named spinasaponin G.

Compound 1 (SOA1) 3-O-β-D-glucuronopyranosyl-2β,3β-dihydroxyolean-12-ene-23,28-dioic acid-28-O-β-d-glucopyranosyl-(1→4)-α-1-rhannopyranosyl-(1→2)-β-D-fucopyranoside (Spinasaponin C)

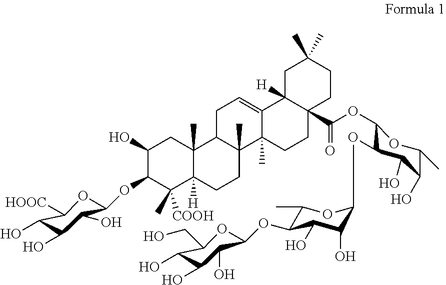

1) Physical properties: Amorphous white crystals
2) Molecular weight: 1133.24
3) Molecular formula: C₅₄H₈₄O₂₅
4) ¹H-NMR (Pyridine-d₅, 900 MHZ) δ_{H} 6.20 (1H, d, J=1.8 Hz, H-1″′, Rham), 5.91 (1H, d, J=8.1 Hz, H-1″, Fuc), 5.33 (1H, s, H-12), 5.13 (1H, d, J=8.1 Hz, H-1′, GlcA), 5.06 (1H, d, J=7.2 Hz, H-1″″, Glu), 4.74 (1H, d, J=2.7 Hz, H-2), 4.63 (1H, d, J=2.7 Hz, H-3), 3.02 (1H, dd, J=13.5, 3.6 Hz, H-18), 2.21 (1H, d, J=12.6 Hz, H-1a), 1.89 (3H, s, H₃-24), 1.64 (3H, d, J=6.3 Hz, H₃-6″′, Rham), 1.45 (3H, s, H₃-25), 1.40 (3H, d, J=6.3 Hz, H₃-6″, Fuc), 1.19 (3H, s, H₃-27), 1.03 (3H, s, H₃-26), 0.79 (3H, s, H₃-30), 0.77 (3H, s, H₃-29); ¹³C-NMR (Pyridine-d₅, 225 MHZ) δ_{C} 44.1 (C-1), 70.0 (C-2), 85.7 (C-3), 52.6 (C-4), 52.3 (C-5), 20.9 (C-6), 32.7 (C-7), 40.1 (C-8), 48.4 (C-9), 36.5 (C-10), 23.7 (C-11), 122.4 (C-12), 143.8 (C-13), 42.0 (C-14), 28.0 (C-15), 23.1 (C-16), 46.8 (C-17), 41.8 (C-18), 46.0 (C-19), 30.4 (C-20), 33.6 (C-21), 32.0 (C-22), 180.9 (C-23), 13.8 (C-24), 16.6 (C-25), 17.2 (C-26), 25.8 (C-27), 176.6 (C-28), 32.8 (C-29), 23.5 (C-30), 105.2 (C-1′, GlcA), 74.4 (C-2′, GlcA), 77.2 (C-3′, GlcA), 72.8 (C-4′, GlcA), 76.9 (C-5′, GlcA), 172.7 (C-6′, GlcA), 94.5 (C-1″, Fuc), 74.1 (C-2″, Fuc), 75.8 (C-3″, Fuc), 72.6 (C-4″, Fuc), 72.1 (C-5″, Fuc), 16.5 (C-6″, Fuc), 101.1 (C-1″′, Rham), 71.2 (C-2″′, Rham), 71.9 (C-3″′, Rham), 84.5 (C-4″′, Rham), 68.2 (C-5″′, Rham), 18.3 (C-6″′, Rham), 106.2 (C-1″″, Glu), 75.9 (C-2″″, Glu), 78.1 (C-3″″, Glu), 71.3 (C-4″″, Glu), 77.9 (C-5″″, Glu), 62.4 (C-6″″, Glu).

Compound 2 (SOA2) 3-O-β-D-xylopyranosyl-(1→3)-β-d-glucuronopyranosyl-2β,3β,-dihydroxyolean-12-ene-23,28-dioic acid-28-O-β-d-glucopyranosyl-(1→4)-α-1-rhamnopyranosyl-(1→2)-β-D-fucopyranoside (Spinasaponin D)

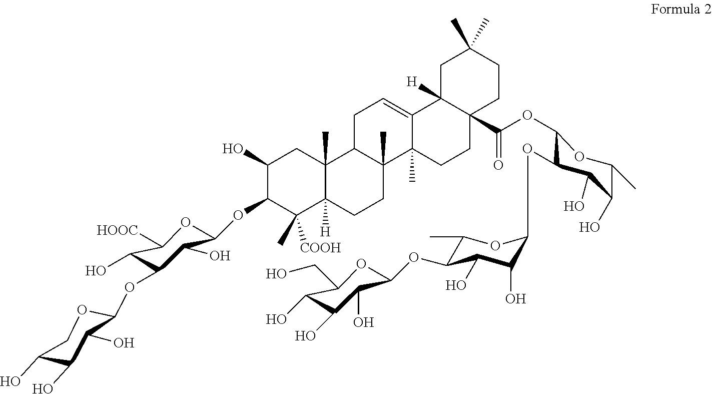

1) Physical properties: Amorphous white crystals
2) Molecular weight: 1265.36
3) Molecular formula: C₅₉H₉₂O₂₉
4) ¹H-NMR (Pyridine-d₅, 800 MHZ) δ_{H} 6.36 (1H, s, H-1″″, Rham), 6.04 (1H, d, J=8.0 Hz, H-1″′, Fuc), 5.46 (1H, br s, H-12), 5.30 (1H, d, J=7.2 Hz, H-1″, Xyl), 5.28 (1H, d, J=8.0 Hz, H-1′, GlcA), 5.17 (1H, d, J=8.0 Hz, H-1″″′, Glu), 4.84 (1H, br s, H-2), 4.77 (1H, br s, H-3), 3.12 (1H, d, J=13.6, 3.2 Hz, H-18), 2.26 (1H, d, J=12.0 Hz, H-1a), 2.01 (3H, s, H₃-24), 1.70 (3H, d, J=5.6 Hz, H₃-6″″, Rham), 1.58 (3H, s, H₃-25), 1.48 (3H, d, J=6.4 Hz, H₃-6″′, Fuc), 1.26 (3H, s, H₃-27), 1.14 (3H, s, H₃-26), 0.90 (3H, s, H₃-30), 0.85 (3H, s, H₃-29); ¹³C-NMR (Pyridine-d₅, 200 MHZ) δ_{C} 44.9 (C-1), 71.0 (C-2), 86.8 (C-3), 53.3 (C-4), 53.1 (C-5), 21.6 (C-6), 33.4 (C-7), 40.8 (C-8), 49.0 (C-9), 37.3 (C-10), 24.3 (C-11), 123.1 (C-12), 144.3 (C-13), 42.7 (C-14), 28.6 (C-15), 23.8 (C-16), 47.3 (C-17), 42.5 (C-18), 46.6 (C-19), 31.1 (C-20), 34.3 (C-21), 32.7 (C-22), 180.9 (C-23), 14.5 (C-24), 17.3 (C-25), 17.8 (C-26), 26.5 (C-27), 177.0 (C-28), 33.5 (C-29), 24.2 (C-30), 106.3 (C-1′, GlcA), 74.4 (C-2′, GlcA), 86.1 (C-3′, GlcA), 71.8 (C-4′, GlcA), 77.0 (C-5′, GlcA), 170.6 (C-6′, GlcA), 106.5 (C-1″, Xyl), 75.8 (C-2″, Xyl), 78.4 (C-3″, Xyl), 71.8 (C-4″, Xyl), 67. 7 (C-5″, Xyl), 95.1 (C-1″′, Fuc), 74.7 (C-2″′, Fuc), 76.8 (C-3″′, Fuc), 73.5 (C-4″′, Fuc), 72.7 (C-5″′, Fuc), 17.2 (C-6″′, Fuc), 101.8 (C-1″″, Rham), 72.1 (C-2″″, Rham), 72.8 (C-3″″, Rham), 85.8 (C-4″″, Rham), 68.9 (C-5″″, Rham), 19.0 (C-6″″, Rham), 107.4 (C-1″″′, Glu), 76.8 (C-2″″′, Glu), 79.2 (C-3″″′, Glu), 72.2 (C-4″″′, Glu), 78.8 (C-5″″′, Glu), 63.3 (C-6″″′, Glu).

Compound 3 (SOA3) 3-O-β-D-glucuronopyranosyl-2β,3β,-dihydroxyolean-12-ene-23,28-dioic acid-28-O-β-d-xylopyranosyl-(1→4)-α-1-rhamnopyranosyl-(1→2)-β-D-fucopyranoside (Celosin I)

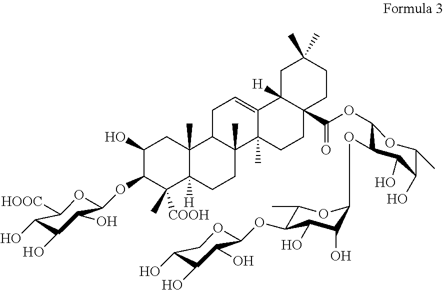

1) Physical properties: Amorphous white crystals
2) Molecular weight: 1103.22
3) Molecular formula: C₅₃H₈₂O₂₄
4) ¹H-NMR (Pyridine-d₅, 900 MHZ) δ_{H} 6.41 (1H, br s, H-1″′, Rham), 6.04 (1H, d, J=8.1 Hz, H-1″, Fuc), 5.41 (1H, s, H-12), 5.22 (1H, br s, H-1′, GlcA), 5.03 (1H, d, J=7.2 Hz, H-1″″, Xyl), 4.85 (1H, m, H-2), 4.75 (1H, m, H-3), 3.12 (1H, d, J=12.6 Hz, H-18), 2.31 (1H, br s, H-1a), 2.00 (3H, s, H₃-24), 1.70 (3H, d, J=6.3 Hz, H₃-6″′, Rham), 1.57 (3H, s, H₃-25), 1.48 (3H, d, J=5.4 Hz, H₃-6″, Fuc), 1.26 (3H, s, H₃-27), 1.14 (3H, s, H₃-26), 0.89 (3H, s, H₃-30), 0.84 (3H, s, H₃-29); ¹³C-NMR (Pyridine-d₅, 225 MHZ) δ_{C} 44.9 (C-1), 70.7 (C-2), 86.7 (C-3), 53.3 (C-4), 52.9 (C-5), 21.6 (C-6), 33.4 (C-7), 40.8 (C-8), 49.0 (C-9), 37.2 (C-10), 24.3 (C-11), 123.1 (C-12), 144.3 (C-13), 42.7 (C-14), 28.6 (C-15), 23.7 (C-16), 47.4 (C-17), 42.4 (C-18), 46.6 (C-19), 31.1 (C-20), 34.3 (C-21), 32.7 (C-22), 181.0 (C-23), 14.6 (C-24), 17.3 (C-25), 17.8 (C-26), 26.4 (C-27), 177.0 (C-28), 33.5 (C-29), 24.1 (C-30), 106.0 (C-1′, GlcA), 75.3 (C-2′, GlcA), 78.2 (C-3′, GlcA), 73.7 (C-4′, GlcA), 77.6 (C-5′, GlcA), 173.0 (C-6′, GlcA), 95.1 (C-1″, Fuc), 74.4 (C-2″, Fuc), 77.0 (C-3″, Fuc), 73.5 (C-4″, Fuc), 72.7 (C-5″, Fuc), 17.3 (C-6″, Fuc), 101.7 (C-1″′, Rham), 71.3 (C-2″′, Rham), 72.9 (C-3″′, Rham), 85.8 (C-4″′, Rham), 68.6 (C-5″′, Rham), 18.9 (C-6″′, Rham), 108.0 (C-1″″, Xyl), 76.6 (C-2″″, Xyl), 79.2 (C-3″″, Xyl), 72.2 (C-4″″, Xyl), 67.9 (C-5″″, Xyl).

Compound 4 (SOA4) 3-O-β-D-xylopyranosyl-(1→3)-β-D-glucuronopyranosyl-2β,3β-dihydroxyolean-12-ene-23,28-dioic acid-28-O-β-D-xylopyranosyl-(1→4)-α-L-rhamnopyranosyl-(1→2)-β-D-fucopyranoside (Spinasaponin E)

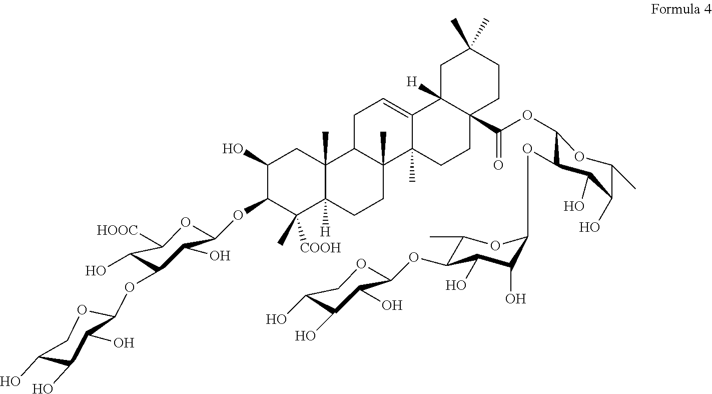

1) Physical properties: Amorphous white crystals
2) Molecular weight: 1235.33
3) Molecular formula: C₅₈H₉₀O₂₈
4) ¹H-NMR (Pyridine-d₅, 900 MHZ) δ_{H} 6.25 (1H, s, H-1″″, Rham), 5.91 (1H, d, J=8.1 Hz, H-1″′, Fuc), 5.34 (1H, br s, H-12), 5.16 (1H, d, J=8.1 Hz, H-1′, GlcA), 5.15 (1H, d, J=8.1 Hz, H-1″, Xyl-I), 4.94 (1H, d, J=7.2 Hz, H-1″″′, Xyl-II), 4.72 (1H, br s, H-2), 4.65 (1H, d, J=3.6 Hz, H-3), 3.02 (1H, dd, J=13.5, 3.6 Hz, H-18), 2.18 (1H, d, J=12.6 Hz, H-1a), 1.88 (3H, s, H₃-24), 1.60 (3H, d, J=6.3 Hz, H₃-6″″, Rham), 1.44 (3H, s, H₃-25), 1.40 (3H, d, J=6.3 Hz, H₃-6″′, Fuc), 1.19 (3H, s, H₃-27), 1.03 (3H, s, H₃-26), 0.78 (3H, s, H₃-30), 0.76 (3H, s, H₃-29); ¹³C-NMR (Pyridine-d₅, 200 MHZ) δ_{C} 44.1 (C-1), 70.1 (C-2), 85.7 (C-3), 52.6 (C-4), 52.4 (C-5), 20.9 (C-6), 32.7 (C-7), 40.1 (C-8), 48.4 (C-9), 36.5 (C-10), 23.7 (C-11), 122.4 (C-12), 143.8 (C-13), 42.1 (C-14), 28.0 (C-15), 23.0 (C-16), 46.8 (C-17), 41.8 (C-18), 46.0 (C-19), 30.4 (C-20), 33.6 (C-21), 32.0 (C-22), 180.7 (C-23), 13.8 (C-24), 16.6 (C-25), 17.1 (C-26), 25.8 (C-27), 176.6 (C-28), 32.8 (C-29), 23.5 (C-30), 104.9 (C-1′, GlcA), 73.6 (C-2′, GlcA), 85.5 (C-3′, GlcA), 71.0 (C-4′, GlcA), 76.4 (C-5′, GlcA), 173.5 (C-6′, GlcA), 105.5 (C-1″, Xyl-I), 74.8 (C-2″, Xyl-I), 77.3 (C-3″, Xyl-I), 70.4 (C-4″, Xyl-I), 66.7 (C-5″, Xyl-I), 94.5 (C-1″′, Fuc), 73.8 (C-2″′, Fuc), 76.0 (C-3″′, Fuc), 72.6 (C-4″′, Fuc), 72.1 (C-5″′, Fuc), 16.6 (C-6″′, Fuc), 101.0 (C-1″″, Rham), 71.2 (C-2″″, Rham), 71.9 (C-3″″, Rham), 84.6 (C-4″″, Rham), 68.0 (C-5″″, Rham), 18.2 (C-6″″, Rham), 107.0 (C-1″″′, Xyl-II), 75.7 (C-2″″′, Xyl-II), 78.0 (C-3″″′, Xyl-II), 70.4 (C-4″″′, Xyl-II), 67.0 (C-5″″′, Xyl-II).

Compound 5 (SOA5) 3-O-β-D-glucuronopyranosyl-2β,3β-dihydroxyolean-12-ene-23,28-dioic acid-28-O-β-D-glucopyranosyl-(1→4)-α-L-rhamnopyranosyl-(1→2)-4-O-acetyl-β-D-fucopyranoside (Spinasaponin F)

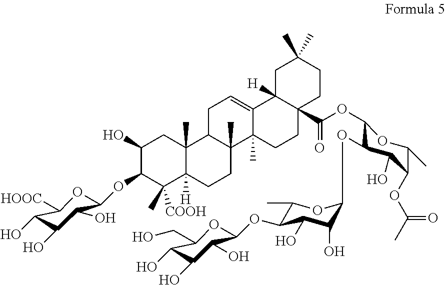

1) Physical properties: Amorphous white crystals
2) Molecular weight: 1175.28
3) Molecular formula: C₅₆H₈₆O₂₆
4) ¹H-NMR (Pyridine-d₅, 900 MHZ) δ_{H} 6.13 (1H, s, H-1″′, Rham), 5.95 (1H, d, J=8.1 Hz, H-1″, Fuc), 5.35 (1H, s, H-12), 5.14 (1H, d, J=7.2 Hz, H-1′, GlcA), 5.07 (1H, d, J=8.1 Hz, H-1″″, Glu), 4.74 (1H, br s, H-2), 4.64 (1H, br s, H-3), 3.02 (1H, dd, J=13.5, 3.6 Hz, H-18), 2.22 (1H, d, J=12.6 Hz, H-1a), 1.95 (3H, s, OAc—CH3), 1.90 (3H, s, H₃-24), 1.72 (3H, d, J=6.3 Hz, H₃-6″′, Rham), 1.46 (3H, s, H₃-25), 1.20 (3H, s, H₃-27), 1.16 (3H, d, J=6.3 Hz, H3-6″, Fuc), 1.02 (3H, s, H3-26), 0.78 (3H, s, H3-30), 0.77 (3H, s, H₃-29); ¹³C-NMR (Pyridine-d₅, 225 MHz) δ_{C} 44.1 (C-1), 70.0 (C-2), 85.7 (C-3), 52.6 (C-4), 52.3 (C-5), 20.9 (C-6), 32.8 (C-7), 40.1 (C-8), 48.4 (C-9), 36.5 (C-10), 23.7 (C-11), 122.4 (C-12), 143.8 (C-13), 42.1 (C-14), 27.9 (C-15), 23.1 (C-16), 46.8 (C-17), 41.7 (C-18), 46.0 (C-19), 30.4 (C-20), 33.6 (C-21), 32.1 (C-22), 180.9 (C-23), 13.8 (C-24), 16.7 (C-25), 17.2 (C-26), 25.8 (C-27), 176.6 (C-28), 32.8 (C-29), 23.5 (C-30), 105.2 (C-1′, GlcA), 74.4 (C-2′, GlcA), 77.1 (C-3′, GlcA), 72.8 (C-4′, GlcA), 77.1 (C-5′, GlcA), 172.0 (C-6′, GlcA), 94.3 (C-1″, Fuc), 74.4 (C-2″, Fuc), 73.3 (C-3″, Fuc), 74.5 (C-4″, Fuc), 70.2 (C-5″, Fuc), 16.2 (C-6″, Fuc), 171.3 (OAc), 20.6 (OAc—CH₃), 101.5 (C-1″′, Rham), 71.1 (C-2″′, Rham), 71.8 (C-3″′, Rham), 84.4 (C-4″′, Rham), 68.4 (C-5″′, Rham), 18.5 (C-6″′, Rham), 106.2 (C-1″″, Glu), 75.8 (C-2″″, Glu), 78.1 (C-3″″, Glu), 71.3 (C-4″″, Glu), 78.1 (C-5″″, Glu), 62.4 (C-6″″, Glu).

Compound 6 (SOA6) 3-O-β-D-xylopyranosyl-(1→3)-β-D-glucuronopyranosyl-2β,3β-dihydroxyolean-12-ene-23,28-dioic acid-28-O-β-D-glucopyranosyl-(1→4)-α-L-rhamnopyranosyl-(1→2)-4-O-acetyl-β-D-fucopyranoside (Spinasaponin G)

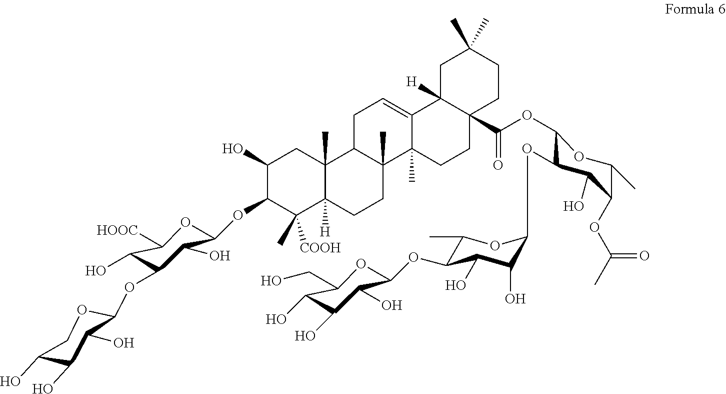

1) Physical properties: Amorphous white crystals
2) Molecular weight: 1307.39
3) Molecular formula: C₆₁H₉₄O₃₀
4) ¹H-NMR (Pyridine-d₅, 900 MHZ) δ_{H} 6.13 (1H, s, H-1″″, Rham), 5.94 (1H, d, J=8.1 Hz, H-1″′, Fuc), 5.34 (1H, s, H-12), 5.15 (1H, d, J=7.2 Hz, H-1″, Xyl), 5.16 (1H, d, J=7.2 Hz, H-1′, GlcA), 5.06 (1H, d, J=8.1 Hz, H-1″″′, Glu), 4.72 (1H, br s, H-2), 4.65 (1H, br s, H-3), 3.02 (1H, dd, J=13.5, 2.7 Hz, H-18), 2.19 (1H, d, J=12.6 Hz, H-1a), 1.94 (3H, s, OAc—CH₃), 1.89 (3H, s, H₃-24), 1.72 (3H, d, J=6.3 Hz, H₃-6″″, Rham), 1.46 (3H, s, H₃-25), 1.20 (3H, s, H₃-27), 1.16 (3H, d, J=6.3 Hz, H₃-6″′, Fuc), 1.02 (3H, s, H₃-26), 0.81 (3H, s, H₃-30), 0.77 (3H, s, H₃-29); ¹³C-NMR (Pyridine-d₅, 225 MHZ) δ_{C} 44.1 (C-1), 70.1 (C-2), 85.7 (C-3), 52.6 (C-4), 52.4 (C-5), 20.9 (C-6), 32.7 (C-7), 40.1 (C-8), 48.4 (C-9), 36.5 (C-10), 23.7 (C-11), 122.4 (C-12), 143.8 (C-13), 42.1 (C-14), 27.9 (C-15), 23.2 (C-16), 46.8 (C-17), 41.7 (C-18), 46.0 (C-19), 30.4 (C-20), 33.6 (C-21), 32.0 (C-22), 180.7 (C-23), 13.8 (C-24), 16.6 (C-25), 17.2 (C-26), 25.8 (C-27), 176.6 (C-28), 32.8 (C-29), 23.5 (C-30), 105.0 (C-1′, GlcA), 73.6 (C-2′, GlcA), 85.6 (C-3′, GlcA), 73.3 (C-4′, GlcA), 75.7 (C-5′, GlcA), 173.9 (C-6′, GlcA), 105.5 (C-1″, Xyl), 74.8 (C-2″, Xyl), 77.3 (C-3″, Xyl), 70.4 (C-4″, Xyl), 66.7 (C-5″, Xyl), 94.3 (C-1″′, Fuc), 74.4 (C-2″′, Fuc), 73.6 (C-3″′, Fuc), 74.5 (C-4″′, Fuc), 70.2 (C-5″′, Fuc), 16.2 (C-6″′, Fuc), 171.2 (OAc), 20.6 (OAc—CH3), 101.5 (C-1″″, Rham), 71.1 (C-2″″, Rham), 71.8 (C-″″, Rham), 84.4 (C-4″″, Rham), 68.4 (C-5″″, Rham), 18.4 (C-6″″, Rham), 106.2 (C-1″″′, Glu), 75.8 (C-2″″′, Glu), 78.0 (C-3″″′, Glu), 71.2 (C-4″″′, Glu), 77.9 (C-5″″′, Glu), 62.3 (C-6″″′, Glu).

### Example 4

### In Vitro Evaluation of Inflammatory Index Improvement Efficacy of a Spinach Extract, Fractions, Five (5) Novel Substances, and Celosin I Compound

In order to evaluate the effect of a spinach extract, fractions (butanol fraction, Fr.1 and Fr.2), five (5) novel substances (SOA1, SOA2, SOA4, SOA5, SOA6) and celosin I compound (SOA 3) prepared/obtained through Examples 1 to 3 for improving inflammatory indicators, macrophages (RAW264.7) were treated with LPS and treated with each of the test substances at the concentrations indicated in the figures. Then, the cell culture fluid was collected, and the amount of inflammatory cytokines was measured by ELISA according to a previously reported method.

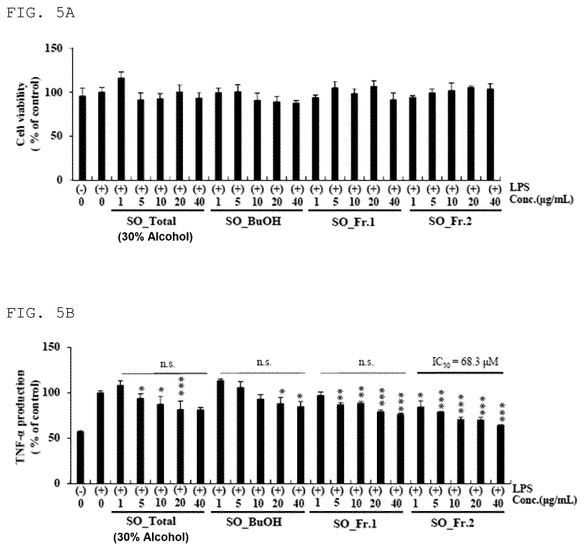

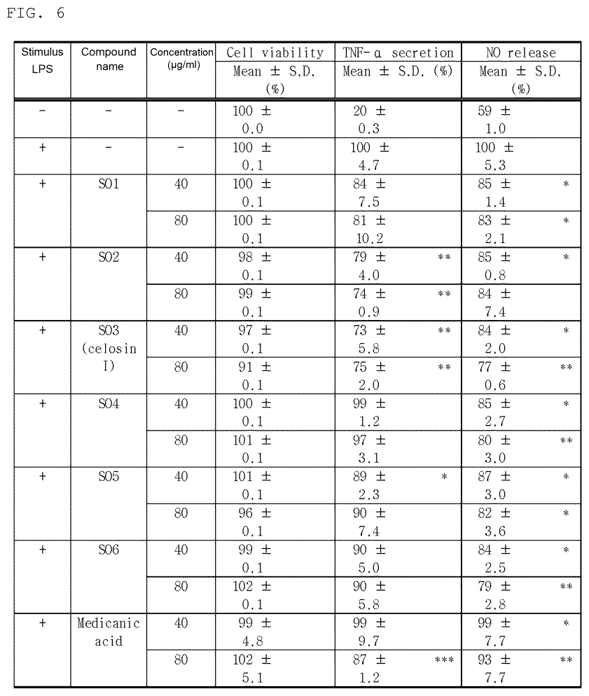

As shown in FIGS. 5A and 5B, it was confirmed that LPS increased the secretion of TNF-alpha in macrophages, but the secretion of TNF-alpha decreased in the experimental group treated with the spinach extract or fractions at a non-cytotoxic concentration (FIG. 5B).

In particular, it was confirmed that Fr.2 fraction comprising the novel glycosides of formulae 1 to 6 more effectively inhibited the secretion of TNF-alpha induced by LPS, as compared to the spinach extract, butanol fraction and Fr.1.

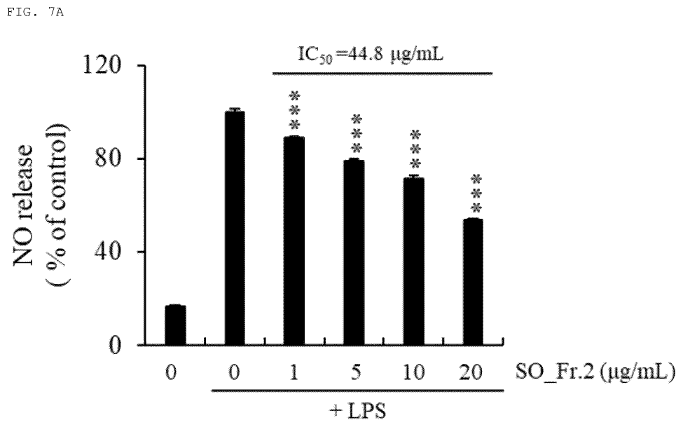

As shown in FIG. 6, it was confirmed that the novel glycoside compounds of Formulae 1 to 6 more effectively inhibited the secretion of TNF-alpha and nitric oxide (NO) in macrophages by LPS, as compared to medicagenic acid (MA).

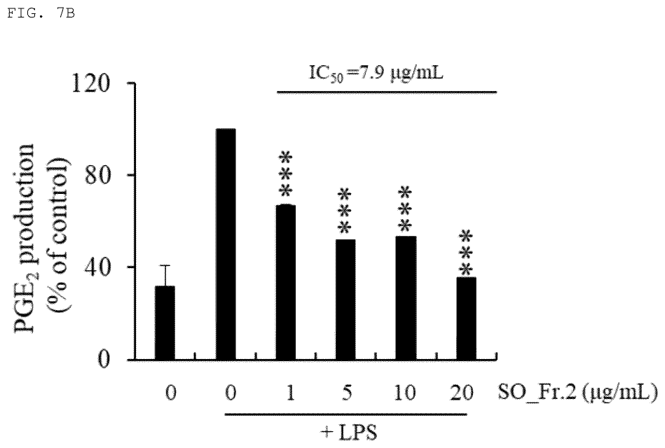

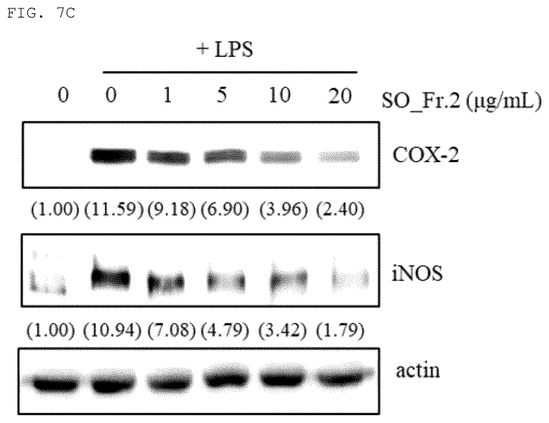

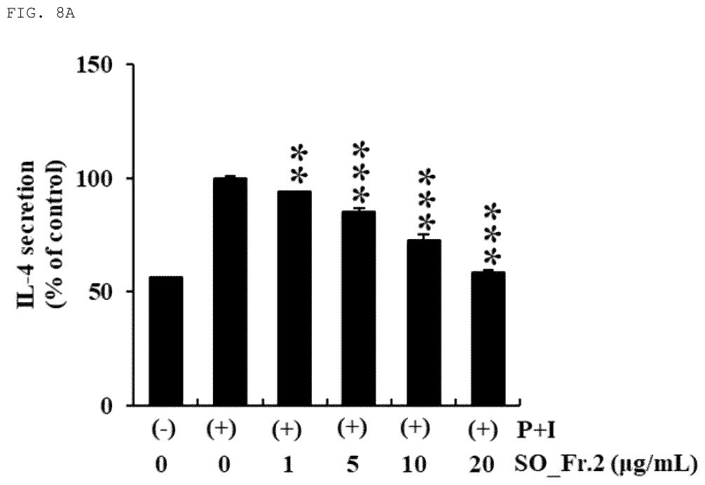

Meanwhile, as shown in FIGS. 7A to 7C, it was confirmed that Fr.2 comprising the novel glycoside compounds of Formulae 1 to 6 inhibited the secretion of NO and PEG2 in macrophages treated with LPS in a concentration-dependent manner (FIGS. 7A and 7B), and also inhibited the expression of COX-2 and iNOS proteins in a concentration-dependent manner (FIG. 7C).

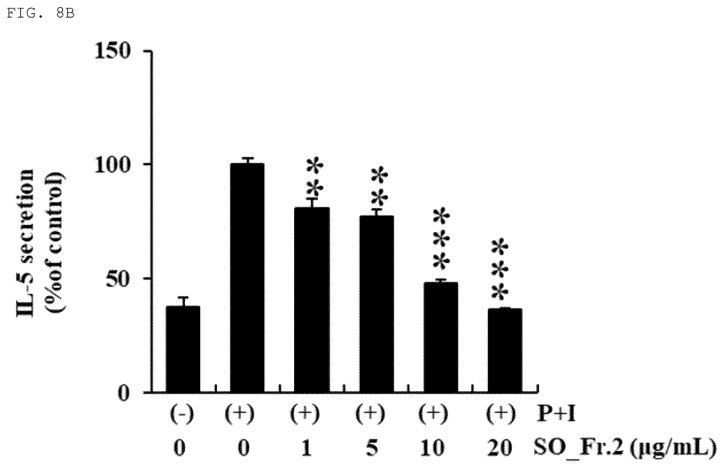

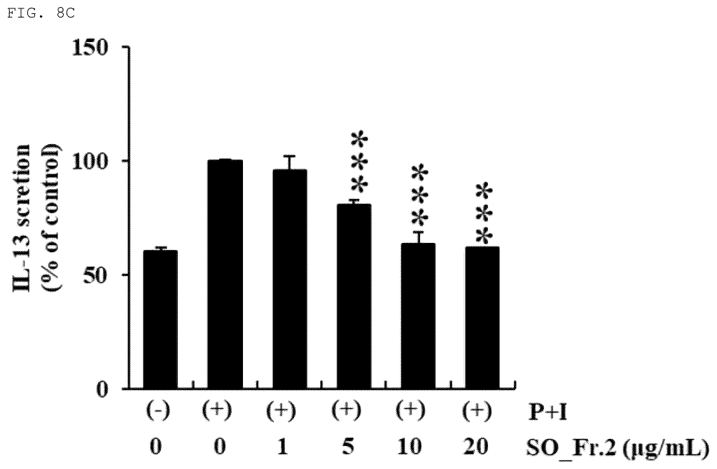

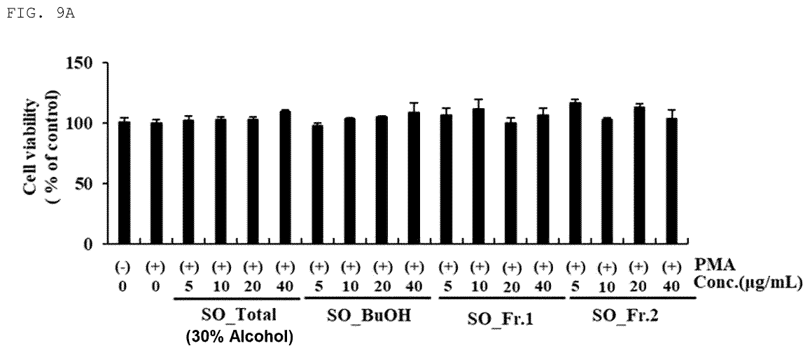

In addition, as a result of stimulating EL4 cells with PMA and lonomycin and treating them with Fr.2 fraction of the spinach extract, as shown in FIGS. 8A to 8C, it was confirmed that the secretion of inflammatory cytokines (IL-4, IL-5 and IL-13) was inhibited in a concentration-dependent manner.

### Example 5

### Evaluation of Effect on MUC5AC Secretion in Respiratory Epithelial Cells by PMA Stimulation

In order to confirm whether the spinach extract, fractions (butanol fraction, Fr.1 and Fr.2) and five (5) novel substances (SOA1, SOA2, SOA4, SOA5, SOA6) and celosin I compound (SOA 3) prepared/obtained through Examples 1 to 3 may control the excessive secretion of mucus in inflammatory respiratory diseases, respiratory epithelial cells NCI-H292 were stimulated with PMA, and treated with each of the test substances at the concentrations indicated in the figures. Next, the amount of MUC5AC secreted from the cells was measured.

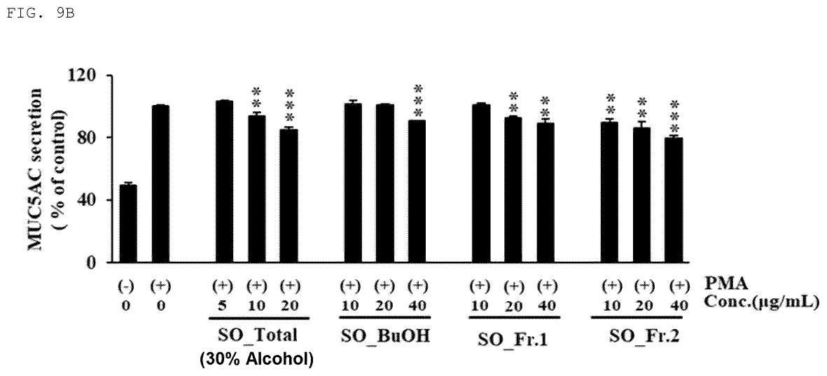

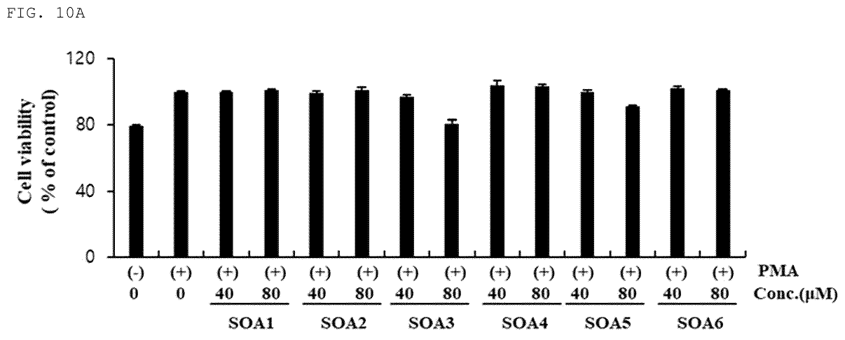

As a result, as shown in FIGS. 9A and 9B, the effect of Fr.2 fraction on inhibition of mucus secretion from respiratory epithelial cells was significantly higher, as compared to the spinach extract, butanol fraction, and Fr.1 fraction (FIG. 9B).

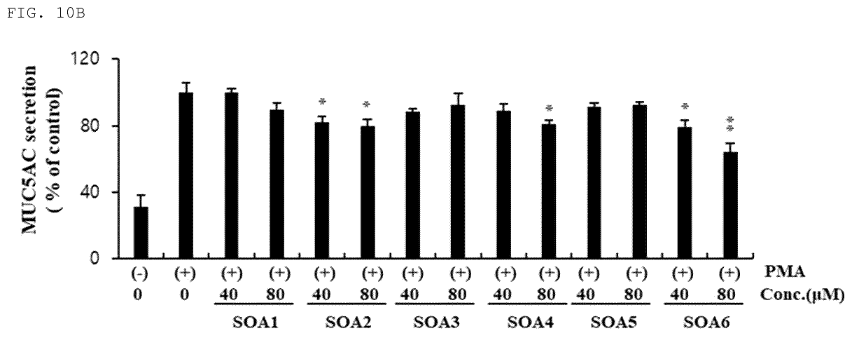

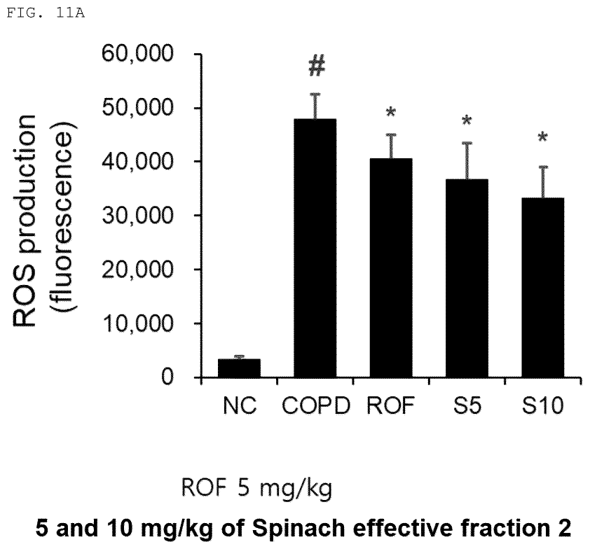

Meanwhile, the saponin compounds of Formulae 1 to 6 comprising novel glycoside compounds also very effectively inhibited mucus secretion from respiratory epithelial cells caused by PMA stimulation (FIGS. 10A and 10B).

### Example 6

### Evaluation of Efficacy in Inflammatory Respiratory Disease Animal Models

To confirm the anti-COPD effect of effective fractions of spinach in COPD mouse models, COPD mouse models in which COPD was induced by cigarette smoke and lipopolysaccharide were prepared. In order to confirm the effect of the effective fractions of spinach for inhibiting ROS, elastase, TNF-α, IL-6 in bronchoalveolar lavage fluid in COPD mice, the produced amount of each of them was analyzed by using ELISA assay technique.

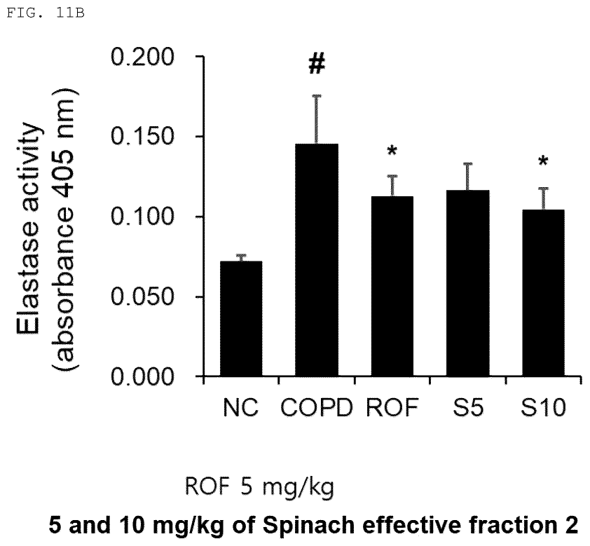

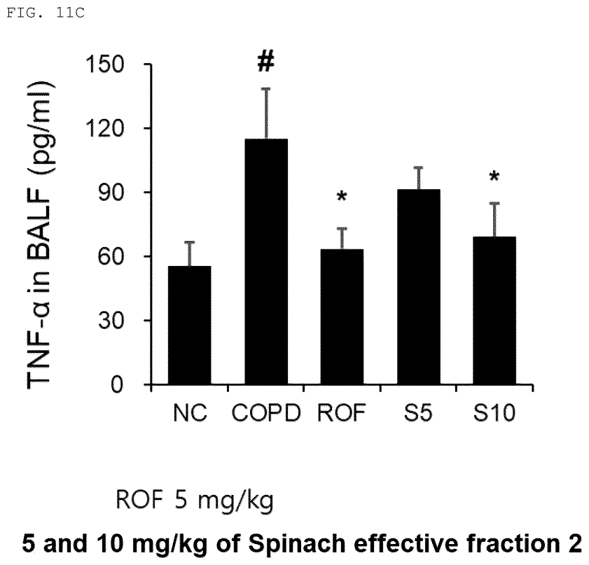

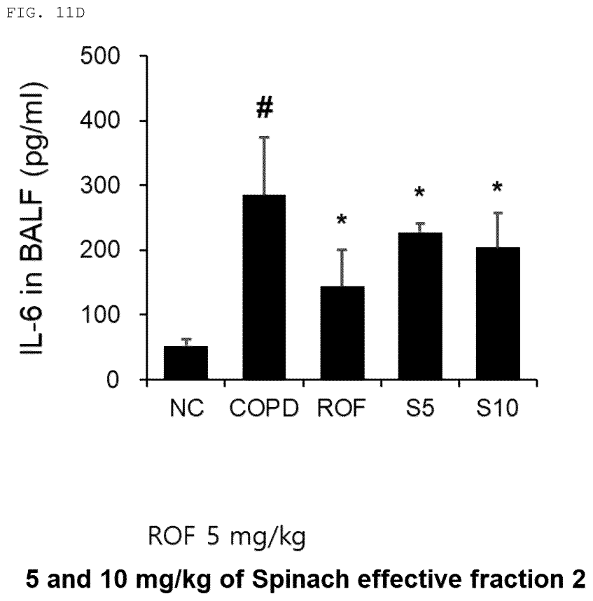

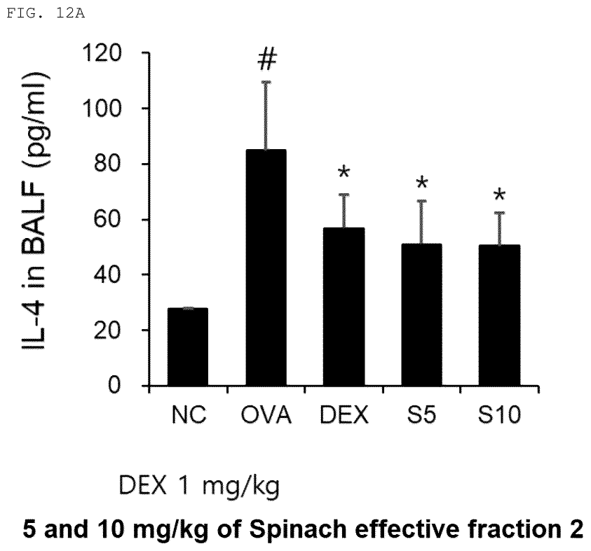

As a result, as shown in FIGS. 11A to 11D, it was confirmed that the levels of ROS, elastase, TNF-α, and IL-6 in the bronchoalveolar lavage fluid of experimental group S10 administered orally with the prepared effective fraction of spinach (Fr.2), was significantly decreased, as compared to the COPD-induced group, and, through this, it was confirmed that the effective fraction of spinach (Fr.2) were effective for inhibiting inflammatory cytokines, ROS and elastase in COPD mice.

In order to confirm the anti-asthmatic effect of the effective fraction of spinach (Fr.2) in asthma mouse models, asthma mouse models in which bronchial asthma was induced by using ovalbumin were prepared.

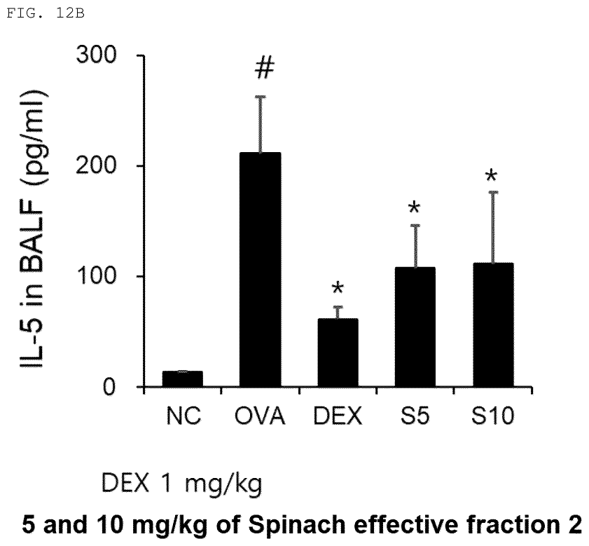

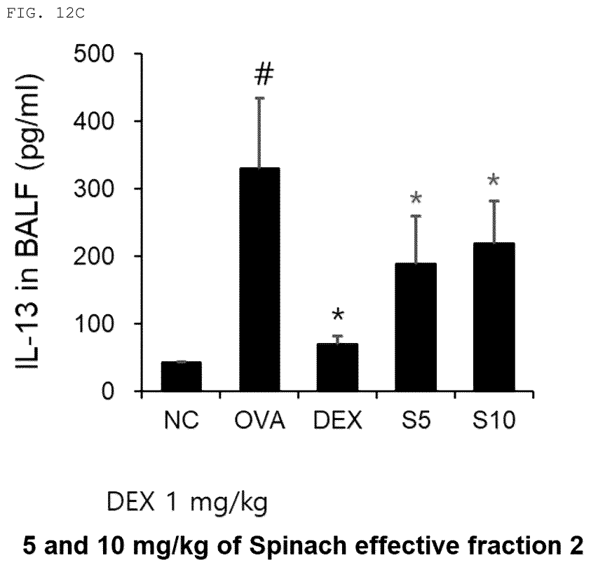

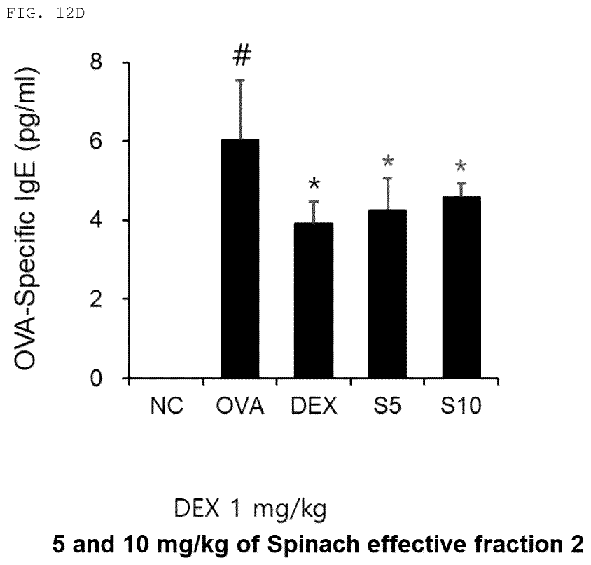

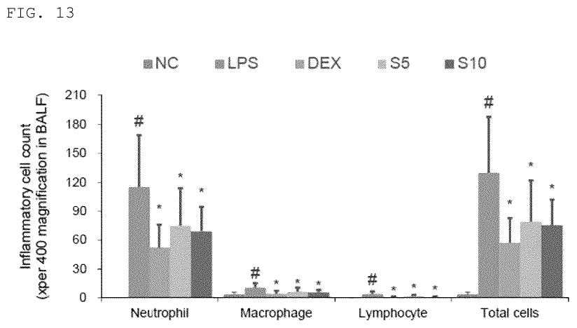

As a result, as shown in FIGS. 12A to 12D, the levels of IL-4, IL-5, IL-13 in the bronchoalveolar lavage fluid of experimental groups S5 and S10 administered orally with the effective fraction of spinach (Fr.2) were significantly decreased, as compared to the asthma-induced group. In addition, the serum IgE levels of experimental groups S5 and S10 administered orally with the effective fraction of spinach (Fr.2) were significantly decreased, as compared to the asthma-induced group. Through this, it was confirmed that the effective fraction of spinach (Fr.2) were effective for inhibiting Th2 cytokines and IgE in asthmatic mice.

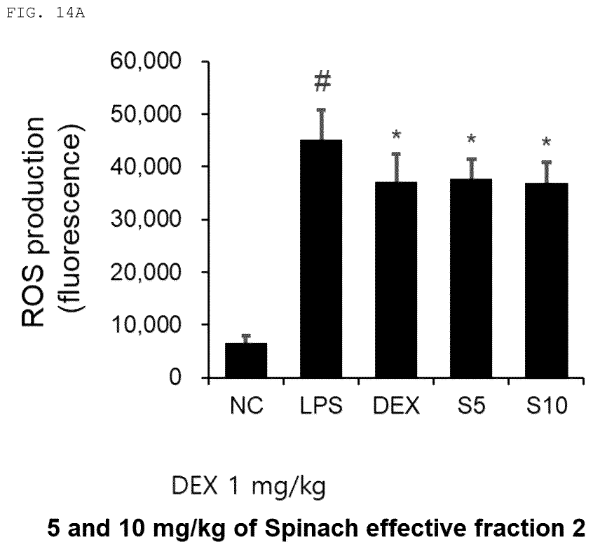

In order to confirm the anti-pneumonia effect of the effective fraction of spinach (Fr.2) in pneumonia mouse models, pneumonia mouse models in which bronchopneumonia was induced by using lipopolysaccharide were prepared. The increased influx of neutrophils and macrophages is an important feature of the mechanism of pneumonia development and, therefore, in order to confirm the inhibitory effect of spinach effective fraction (Fr.2) on inflammatory cells in bronchoalveolar lavage fluid in mice with pneumonia, each of the cells was isolated and the number of cells were measured by using Diff quik® staining (FIG. 13).

As a result, it was confirmed that the number of neutrophils and macrophages was significantly increased in the bronchoalveolar lavage fluid of mice with pneumonia induced by lipopolysaccharide, and the experimental groups S5 and S10 administered orally with the effective fraction of spinach were effective for decreasing the number of inflammatory cells.

In order to confirm the effect of the effective fraction of spinach (Fr.2) for inhibiting ROS, TNF-α and IL-6 in bronchoalveolar lavage fluid in mice with pneumonia, the produced amount of each of them was quantitatively analyzed by using ELISA assay technique.

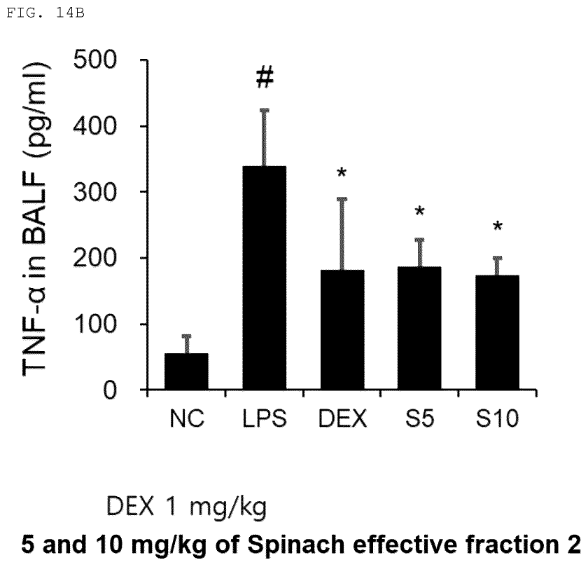

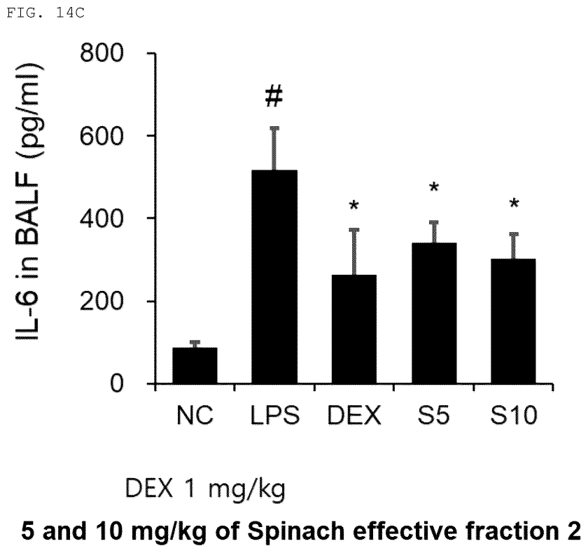

As a result, as shown in FIGS. 14A to 14C, it was

confirmed that the levels of a, b, and c in the bronchoalveolar lavage fluid of experimental groups S5 and S10 administered orally with the effective fraction of spinach (Fr.2) were significantly decreased, as compared to the pneumonia-induced group, and, through this, the effective fraction of spinach was effective for inhibiting ROS and inflammatory cytokines in mice with pneumonia.

### INDUSTRIAL APPLICABILITY

The novel glycoside compound and the spinach extract fraction comprising same provided by the present invention are effective for effectively inhibiting the secretion of excessive inflammatory cytokines from immune cells and suppressing excessive secretion of mucus in inflammatory respiratory diseases such that they may be used in preventing or treating various inflammatory diseases such as inflammatory respiratory diseases. Therefore, the present invention has a high industrial applicability.

1. A compound represented by any one of the following formulae 1 to 6: 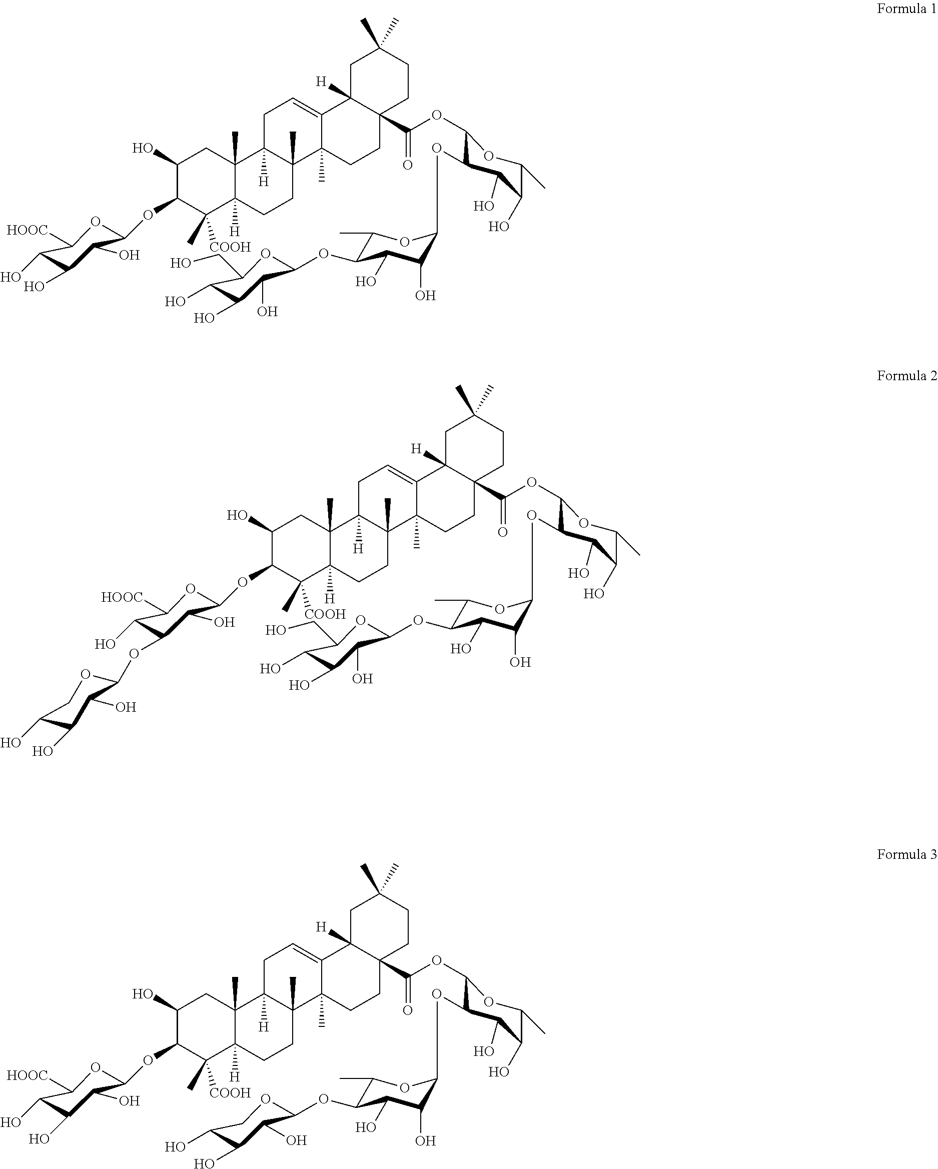 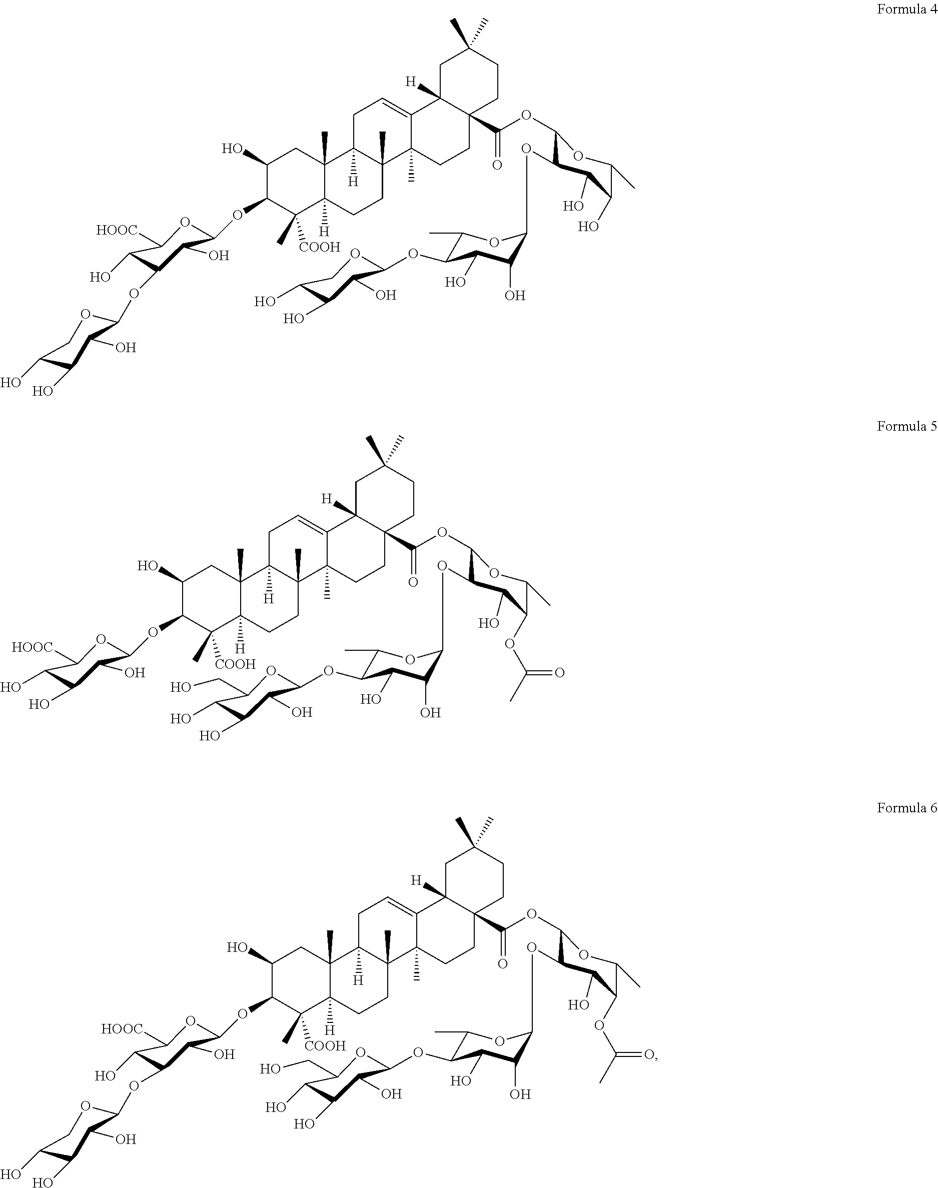
pharmaceutically acceptable salts thereof, isomers thereof, hydrates thereof or solvates thereof.

2. The compound of claim 1, wherein the compound is isolated from spinach.

3. A method for preparing the compound of claim 1, comprising:
(a) extracting spinach with a solvent selected from the group consisting of water, organic solvents, subcritical fluids, supercritical fluids and mixtures thereof;
(b) fractionating the spinach extract by chromatography to obtain fractions; and
(c) isolating the compound from the fractions.

4. The method of claim 3, wherein, in step (a), the organic solvent is selected from the group consisting of alcohol, acetone, ether, benzene, chloroform, ethyl acetate, methylene chloride, hexane, cyclohexane and petroleum ether having 1 to 6 carbon atoms.

5. The method of claim 3, wherein, in step (b), the chromatography is sequentially developed according to a concentration gradient by using water and a non-polar solvent as mobile phases.

6. The method of claim 5, wherein, in step (b), fractions fractionated from 20 to 100% (v/v) of a non-polar solvent aqueous solution are obtained.

7. A pharmaceutical composition for preventing or treating inflammatory diseases, comprising as an active ingredient one or more selected from the group consisting of the compounds according to claim 1 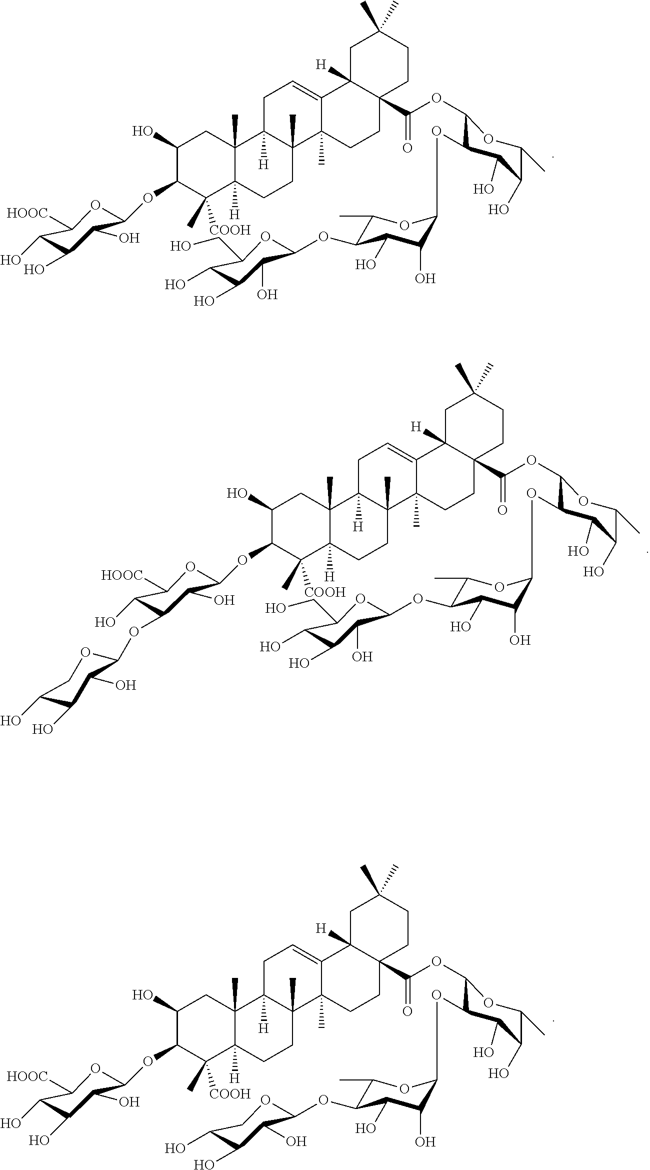 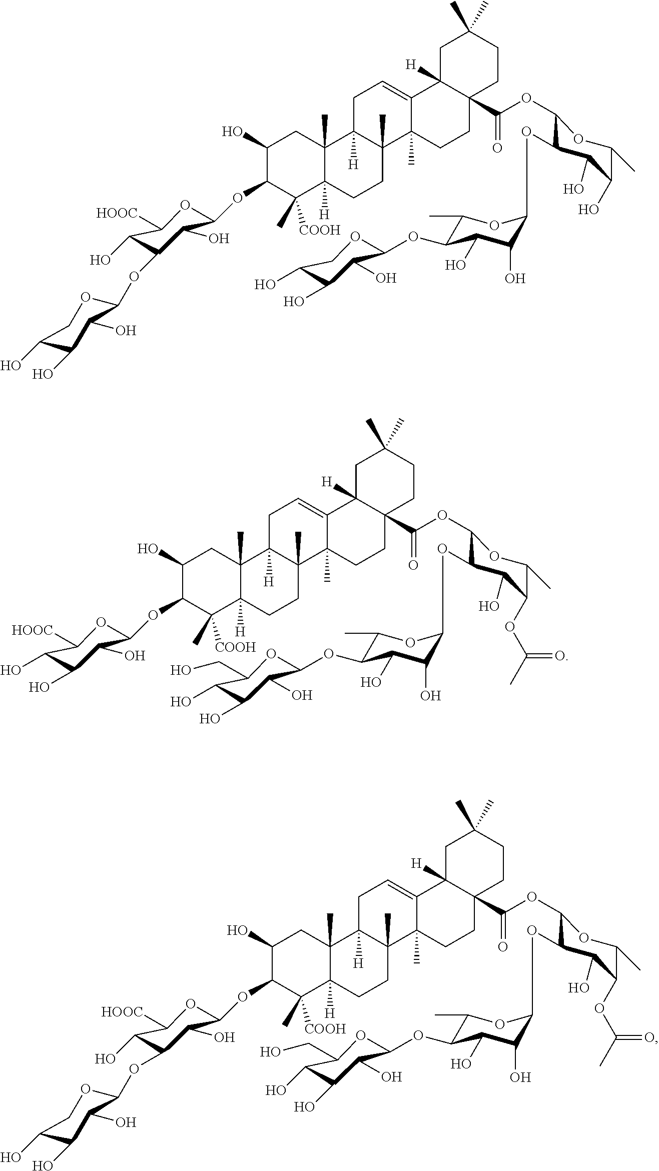
pharmaceutically acceptable salts thereof, isomers thereof, hydrates thereof, and solvates thereof.

8. The pharmaceutical composition of claim 7, wherein the inflammatory disease is selected from the group consisting of inflammatory respiratory disease, dermatitis, atopic dermatitis, allergy, psoriasis, bronchitis, ulcerative colitis, retinitis, uveitis, conjunctivitis, arthritis, rheumatoid arthritis, ankylosing spondylitis, osteoarthritis, nephritis, nephritis, autoimmune pancreatitis, chronic pelvic inflammatory disease, endometritis, otitis media, cystitis, and chronic prostatitis.

9. The pharmaceutical composition of claim 8, wherein the inflammatory respiratory disease is selected from the group consisting of asthma, pneumonia, acute lung injury, acute respiratory failure syndrome, chronic obstructive pulmonary disease, allergic rhinitis, bronchitis, pharyngitis, laryngitis, pharyngitis, and tonsillitis.

10.-16. (canceled).

17. A method of treating inflammatory diseases, comprising administering to a subject in need thereof an effective amount of a composition comprising as an active ingredient one or more selected from the group consisting of the compounds represented by any one of the following formulae 1 to 6: 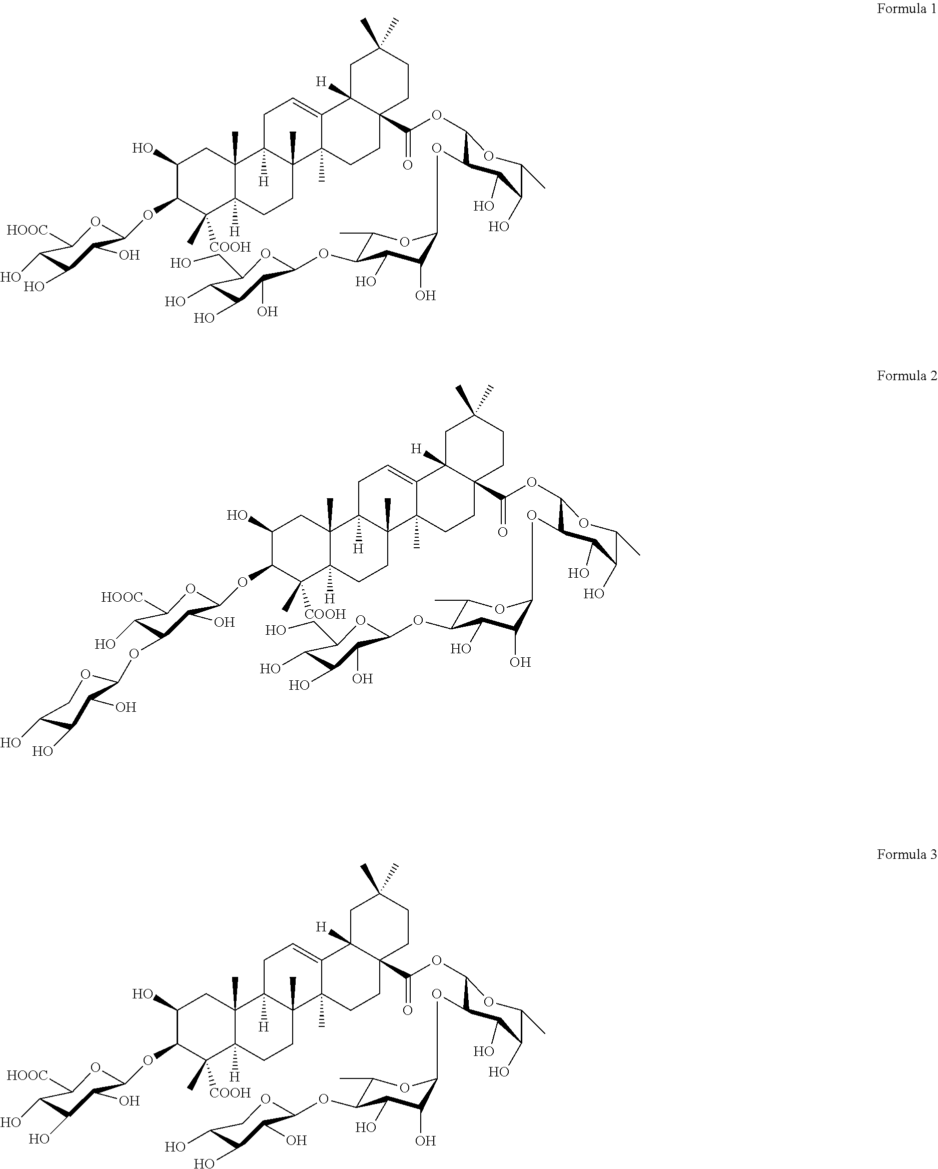 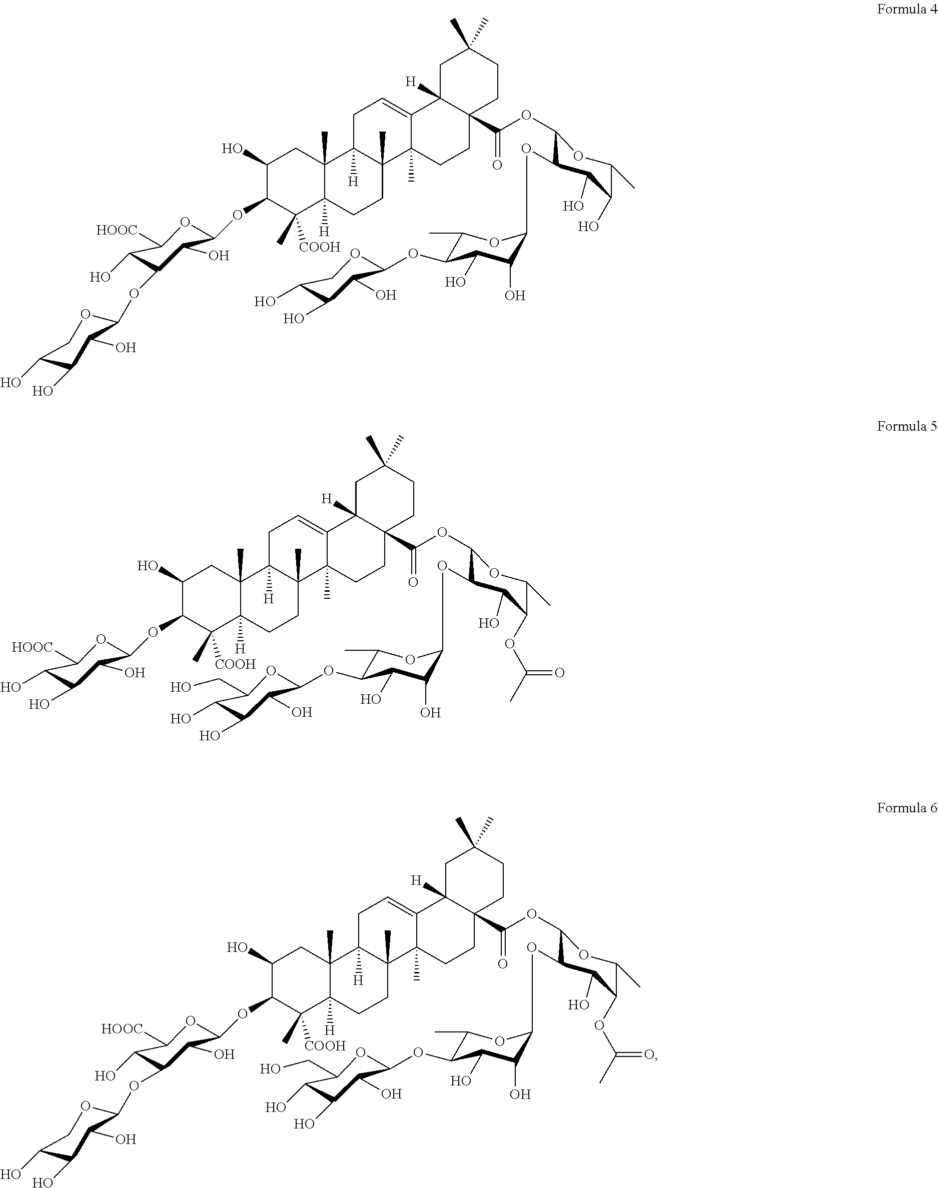
pharmaceutically acceptable salts thereof, isomers thereof, hydrates thereof, and solvates thereof.
